(19) United States
(12) Patent Application Publication
(10) Pub. No.: US 2010/0151092 A1
(43) Pub. Date: Jun. 17, 2010

(54) Steam injection cooking device and method

(76) Inventors: Gerald A. Sus, Frankfort, IL (US); Henry T. Ewald, Roselle, IL (US); Paul G. Simmons, Glen Ellyn, IL (US)

Correspondence Address: RYNDAK & SURI LLP, 200 W. MADISON STREET, SUITE 2100, CHICAGO, IL, 60606, US

(21) Appl. No.: 12/658,519

(22) Filed: Feb. 11, 2010

Related U.S. Application Data
(63) Continuation-in-part of application No. 12/080,850, filed on Apr. 7, 2008.

(51) Int. Cl. A23L 1/01; G01N 33/02

(52) U.S. Cl. 426/231; 426/510

(57) ABSTRACT

An apparatus and method are provided for steam injection heating of a liquid or solid edible material. When cooking, the method includes providing a predetermined amount of edible material. A predetermined amount of water supplied from a water tank is injected into a heated cavity to form a quantity of steam that is sufficient to cook the predetermined quantity of edible material when injected therein. The predetermined amount of water is provided by a liquid water injector. The predetermined amount of the edible material may be a single serving, or integer multiples thereof. When heating a serving amount that is an integer multiple of at least two times the amount of a single serving, the predetermined quantity of water is injected in pulses. The number of pulses is the same as the integer multiple of servings being heated, and each pulse contains substantially equal amounts of liquid water.

### CROSS-REFERENCE TO RELATED APPLICATION

This application is a continuation-in-part application of Ser. No. 12/080,850, filed Apr. 7, 2008, the entire specification of which is incorporated by reference.

### FIELD OF THE INVENTION

This invention relates to a steam injection cooking device and method for heating a food product, which may be a single portion serving quantity of food.

### BACKGROUND OF THE INVENTION

This invention generally relates to a method and apparatus for high speed injection steam heating and cooking of a containerized batch of bulk food product. The apparatus and method are particularly useful in cooking scrambled eggs in quantities typically used in filling orders in a Quick Service Restaurant (QSR).

Scrambled eggs have been commonly cooked in small batches in a frying pan or on a grill. Attempts that have been made to cook scrambled eggs by steam heating have generally been used to cook larger batches of eggs in a continuous production process, rather than a smaller amount, such as for filling an individual serving scrambled egg order, or a few orders, such as is advantageous for QSR operations.

U.S. Pat. No. 4,228,193 to Schindler discloses a method and apparatus for cooking smaller batches of uncooked shelled eggs to form scrambled eggs using compressed air in combination with steam. The apparatus includes an air compressor, compressed air tank, and a steam generator that makes the apparatus relatively bulky. The device is not easily portable or suitable for countertop use.

A need exists for a method for rapidly cooking by steam injection scrambled eggs that is safe, efficient, and relatively easily controlled.

A need also exists for relatively rapidly cooking scrambled eggs in various size batches that are typical order sizes experienced for a QSR.

A need further exists for rapidly heating to a predetermined temperature different types of bulk food products, in smaller amounts such as one or more serving sizes.

A still further need exists for a device for heating a bulk food product that is safe, efficient, portable and self contained, so that the device is suitable for placement and use in a home kitchen, office, or other such locations.

### SUMMARY OF THE INVENTION

In accordance with various aspects of the present invention, devices and methods are provided for heating and/or cooking a food product. The amount of food heated or cooked may be an individual portion or multiple portions, for example. The type of food product typically is a food product that can be heated and/or cooked by impingement of steam onto the food product.

In accordance with one aspect of the present invention, a method of scrambling eggs is provided. The method comprises providing a predetermined amount of bulk liquid uncooked egg. A predetermined amount of water is injected in a heated cavity forming a quantity of steam in the cavity from substantially all of the water injected in the cavity. The steam is injected into the bulk liquid uncooked egg wherein the quantity of steam is sufficient to cook the predetermined quantity of bulk liquid uncooked egg.

In accordance with another aspect of the invention, the bulk liquid uncooked egg is liquid pasteurized whole egg.

In accordance with a further aspect of the invention, the liquid pasteurized whole egg comprises a desired amount of solids.

In accordance with another aspect of the invention, the predetermined quantity of liquid egg is about a single serving of egg.

In accordance with still another aspect of the invention, the predetermined quantity of bulk liquid egg is an integer multiple of at least 2 times an amount of about a single serving size of egg and the predetermined quantity of water is injected in pulses, the number of pulses injected being the same as the integer multiple, and the amount of water in each pulse being about the same quantity.

In accordance with another aspect of the invention, a method of heating food is provided. As used herein, heating includes heating food that has been previously cooked as well as cooking food partially or completely by heating. The method includes providing a container for holding food and placing a quantity of food in the container. A wand having a terminal end is positioned proximate (which can be near but not in contact with, or, alternatively, immersed in or otherwise in contact with) the bulk food. Pulses of water are injected into a heated cavity to generate steam in the heated cavity. Steam from the steam generator is injected from the wand into the bulk food in the container. The temperature of the bulk food is automatically sensed and the steam injection into the food is automatically terminated when a predetermined temperature of the food is reached.

In accordance with another aspect of the invention, the steam from the wand is of sufficient velocity to penetrate the food and provide mixing of the food.

In accordance with another aspect of the invention, the steam from the wand is of a velocity of about 250 ft/sec to about 800 ft/sec to penetrate the food and provide mixing of the food.

In accordance with another aspect of the invention, the steam from the wand is of a velocity of about 400 ft/sec to about 700 ft/sec to penetrate the food and provide mixing of the food.

In accordance with another aspect of the invention, a method of heating food is provided. As used herein, heating includes heating food that has been previously cooked as well as cooking food partially or completely by heating. The method includes providing a container for holding food and placing a quantity of food in the container. A wand having a terminal end is positioned in the bulk food. Pulses of water are injected into a heated cavity to generate steam in the heated cavity. Steam from the steam generator is injected from the wand into the bulk food in the container. The temperature of the bulk food is automatically sensed and the steam injection into the food is automatically terminated when a predetermined temperature of the food is reached.

In accordance with another aspect of the invention, a method of cooking food is provided. The method includes providing a predetermined amount of a bulk food product. A predetermined amount of water is injected into a heated cavity to form a quantity of steam in the cavity from substantially all of the water injected in the cavity. The steam is injected into the bulk food product wherein the quantity of steam is sufficient to cook the predetermined quantity of the bulk food product.

In accordance with still another aspect of the invention, a method of cooking food is provided that cooks a predetermined quantity of bulk food product that is a single-size serving of food, and the predetermined amount of water in the heated cavity is injected as a single pulse of water.

In accordance with a further aspect of the invention, a method of cooking food is provided wherein the predetermined quantity of bulk food product is an integer multiple of at least 2 times an amount of about a single serving size of food and the predetermined quantity of water that is injected in the heated cavity is injected in pulses, the number of pulses injected being the same as the integer multiple, and the amount of water in each pulse being about the same quantity.

In accordance with another aspect of the invention, an apparatus for cooking scrambled eggs is provided. The apparatus includes a steam generator having a cavity for generating steam from liquid water injected into the steam generator. A liquid water injector is connected to a supply of liquid water for injecting a pulse of a predetermined amount of liquid water into the cavity of the steam generator. The predetermined amount of water injected is sufficient so that when substantially all of the injected water is converted into steam the converted steam is sufficient to cook a predetermined quantity of liquid egg. The apparatus includes a container for containing at least a predetermined quantity of liquid egg placed therein. A wand having a steam exit port is positioned in the container with the steam port proximate to (which may be in contact with, immersed in, or in close relation but not immersed in, or in contact with) the liquid egg. The wand is in fluid communication with the steam generator to receive steam generated therefrom for injecting into the liquid egg placed in the container to make scrambled eggs. Other food products including liquid or fluid food products that can be cooked by steam can be cooked in accordance with the present invention.

In accordance with a further aspect of the invention, the apparatus includes mounting structure for removably mounting the container to seal the container in a substantially closed environment wherein the container is pressurizable in response to the injection of steam therein.

The injection of steam pressurizes the container about 15 psig, or a pressure less than about 15 psig.

In accordance with another aspect of the invention, an apparatus for cooking food is provided. The apparatus includes a steam generator for generating steam by injecting pulses of water on a heated surface and a wand in fluid communication with the steam generator. The wand has an exit port for injecting steam therethrough. The apparatus includes a container having an interior for a receiving a quantity of bulk food for cooking by the apparatus and a sealing surface for substantially sealing the container in a substantially closed environment. Mounting structure is included for removably mounting the container in a position relative to the apparatus so that when the container is in the mounted position, the exit port of the wand is immersed in the bulk food and the interior of the container is substantially sealed by the sealing surface. The apparatus has a control for causing the injection of the pulses of water onto the heated surface of the steam generator.

In a further aspect of the invention, the apparatus for cooking food includes a temperature sensor to automatically sense the temperature of food contained in the container when the container is mounted in position on the apparatus. A control receives temperature information sensed by the temperature sensor to automatically terminate the injection of steam into the bulk food when a predetermined elevated temperature of the food is sensed.

In accordance with a further aspect of the invention, the apparatus for cooking scrambled eggs includes a conduit for communicating the steam generated in the steam generator to the wand with the steam being injected into the bulk food over a period of time. The conduit has an inside diameter of about 0.5 inches and a length suitable for the container, which depending on the size of the container, can be a length in the range of from about 2 inches to about 8 inches.

In accordance with another aspect of the invention, an apparatus for cooking food, which may be scrambled eggs, is provided. The apparatus includes a water tank for containing a supply of liquid water, a steam generator having a cavity for generating steam from liquid water injected into the steam generator, a liquid water injector connectable to the supply of liquid water that is contained in the water tank for injecting a pulse of a predetermined amount of liquid water into the cavity of the steam generator. The predetermined amount of liquid water that is injected is sufficient so that when substantially all of the injected water is converted into steam the converted steam is sufficient to cook a predetermined quantity of liquid egg. In addition, the apparatus may include a container for containing at least a predetermined quantity of liquid egg and a wand having a steam exit port positionable in the container with the steam exit port positioned proximate to the liquid egg contained in the container, the wand being in fluid communication with the steam generator to receive steam generated therefrom for injecting into the liquid egg placed in the container to make scrambled eggs. In accordance with one embodiment, the liquid water injector is composed of a measuring chamber having an inlet and an outlet, the inlet connected to the water tank and the outlet connected to the steam generator, the measuring chamber for temporarily containing a predetermined amount of liquid water received from the water tank for subsequent injection as the pulse of liquid water.

In another embodiment, the measuring chamber has a volume capacity for receiving liquid water, and the volume capacity of the measuring chamber is substantially the same as the volume of the predetermined amount of liquid water injected into the cavity as the pulse.

In another embodiment, the liquid water injector is composed of a flow control valve having an inlet in fluid communication with the water tank and an outlet in fluid communication with the steam generator, the flow control valve for providing the pulse of liquid water injected into the cavity of the steam generator by opening the flow control valve to initiate flow of the pulse and then closing the flow control valve after the predetermined amount of liquid water has flowed through the flow control valve.

In another embodiment, the liquid water injector may comprise a pump for pressurizing liquid water supplied from the water tank. The pump can be utilized to inject a pulse composed of a predetermined amount of liquid water into the cavity of the steam generator.

In accordance with a further aspect of the invention, the apparatus for cooking food includes an inlet valve that is openable to allow water to flow from the water tank into the measuring chamber and is closable to prevent fluid flow between the measuring chamber and the water tank. The apparatus includes an outlet valve closable to contain water in the measuring chamber and openable to release the water contained in the measuring chamber for the pulse. The apparatus has a control for automatically opening the inlet valve and automatically closing the outlet valve to allow the predetermined amount of water to fill the measuring chamber, and thereafter opening the outlet valve to inject the predetermined amount of water as a pulse into the steam generator.

In accordance with another aspect of the invention, the apparatus for cooking food includes a sensor for sensing amount of water contained in the measuring chamber and sending the sensed water amount information to the control to allow the control to automatically close the inlet valve to cause the amount of liquid water contained in the measuring chamber to be the predetermined amount of liquid water of the injected pulse.

In accordance with another aspect of the invention, an apparatus for cooking and/or heating a food product, which may be a liquid or solid edible material (which may be particulate or in discrete pieces, such as rice or pasta, with or without a sauce, for example) is provided. Typically, the food product is of a type that can be cooked with steam. The apparatus includes a steam generator having a cavity for generating steam from liquid water injected into the steam generator, a liquid water injector connectable to the supply of liquid water, the liquid water injector including a chamber for containing a predetermined amount of liquid water for injection of the contained predetermined amount of liquid water as a pulse of the predetermined amount of liquid water into the cavity of the steam generator, the predetermined amount of liquid water injected being sufficient so that when substantially all of the predetermined amount of water is converted into steam, the converted steam is sufficient to cook a predetermined quantity of the liquid or solid edible material. In addition, a container may be provided for containing at least a predetermined quantity of edible material placed therein and a wand having a steam exit port positionable in the container with the steam exit port positioned proximate to the predetermined quantity of a liquid or solid edible material, the wand being in fluid communication with the steam generator to receive steam generated therefrom for injection into the liquid or solid edible material placed in the container to cook the predetermined quantity of the liquid or solid edible material.

In another embodiment, the amount of liquid water contained in the measuring chamber when filled to capacity is substantially the same as the predetermined amount of liquid water injected as a pulse into the cavity of the steam generator.

In accordance with another embodiment of the invention, an apparatus for heating a food product, which can be, for example, a liquid or solid edible material, is provided. The apparatus includes a steam generator for generating steam by injecting pulses of water onto a heated surface, a wand in fluid communication with the steam generator, the wand having an exit port for injecting steam therethrough, a container having an interior for receiving a quantity of a food product to be heated, a sealing surface for substantially sealing the container in a substantially closed environment, a mounting structure for removably mounting the container in a mounted position relative to the wand so that when the container is in the mounted position, the exit port of the wand can be positioned proximate to the food product and the interior of the container is substantially sealed by the sealing surface. In addition, a control for causing the injection of the pulses of water onto the heated surface of the steam generator is provided. The apparatus may further include a measuring chamber for receiving and temporarily containing a predetermined amount of liquid water in the measuring chamber for subsequent injection as the injected pulse to the steam generator.

In a further aspect of the invention, the apparatus for cooking or heating a food product may further include an inlet valve movable between an open and a closed position and an outlet valve moveable between an open and a closed position, a control for automatically controlling the opening and closing of the inlet valve and the outlet valve, the control automatically closing the outlet valve and opening the inlet valve to allow the filling and containing of water in the measuring chamber, and the control automatically closing the inlet valve and opening the outlet valve to inject the water contained in the measuring chamber as the injected pulse of water.

In accordance with another aspect of the invention, the measuring chamber has a diameter to height ratio of about 2:1 or higher.

In accordance with another aspect of the invention, the apparatus for heating a liquid or solid edible material includes a timer device for selecting a period of time for the heating of the edible material by the apparatus. The control automatically causes the apparatus to sequentially inject pulses of water onto the heated surface of the steam generator during the selected period of time and at the expiration of the period of time automatically ceases the injection of pulses onto the heated surface.

In another embodiment, the apparatus may include a supply of liquid water and a flow control valve having an inlet in fluid communication with the supply of liquid water and an outlet in fluid communication with the steam generator, the flow control valve opening to initiate the flow of liquid water to the steam generator and then closing after a predetermined amount of liquid water has flowed through the flow control valve to thereby provide the injected pulse of water.

In another embodiment, the apparatus may include a tank for containing a supply of liquid water for providing the water for the pulses of water, and a pump for pressurizing the supply of liquid water.

In addition, the apparatus may include a temperature sensing device for sensing the temperature of the quantity of food product in the container and sending the sensed temperature to the control. The control can operate to cause sequential injection of pulses of water onto the heated surface of the steam generator which is then injected into the container, until the sensed temperature of the quantity of food product reaches a predetermined temperature and thereafter causes ceasing of the injection of pulses onto the heated surface.

In accordance with another aspect of the invention, a method of heating food is provided that includes providing a water tank for containing a supply of water, providing a container for holding food, placing a quantity of food in the container, positioning a wand having a terminal end proximate but not in contact with the food, sequentially injecting pulses of water from the water tank into a heated cavity of a steam generator to generate steam in the heated cavity from the pulses of water injected into the heated cavity, injecting the steam from the steam generator through the wand and into the food in the container, automatically sensing the temperature of the food and automatically terminating the steam injection into the food when a predetermined temperature of the food is reached.

In accordance with a further aspect of the invention, the method of heating food includes providing a measuring chamber having a volume for receiving and containing water from the water tank, filling the measuring chamber with water received from the water tank, containing the water in the filled measuring chamber; and injecting substantially all the water contained in the filled measuring chamber to provide one of the pulses of water injected into the heated cavity of the steam generator.

In accordance with a further aspect of the invention, the method of heating food includes providing a water amount sensor for sensing the amount of water contained in the measuring chamber during filling, sensing the amount of water contained in the measuring chamber during the filling of the measuring chamber, and using the sensed information to automatically stop the filling of the measuring chamber during filling to provide a predetermined amount of water contained in a filled measuring chamber for injection as one of the pulses of water injected into the steam generator.

In accordance with a further aspect of the invention, the method of heating food includes filling the measuring chamber to full capacity and releasing the water in a filled measuring chamber to provide each pulse of water.

In accordance with a further aspect of the invention, the method of heating food includes providing a valve positioned between the water tank and the heated cavity and automatically opening the valve to allow a predetermined amount of water to flow through the valve to provide each of the pulses of injected water before automatically closing the valve.

In accordance with a further aspect of the invention, the valve is a metering valve.

In accordance with a further aspect of the invention, the valve is a flow control valve.

In accordance with a further aspect of the invention, the valve is a solenoid valve and the supply of water to the valve is pressurized.

In accordance with a further aspect of the invention, the pulse of injected water is formed in a shape that has a cross-sectional area to volume ratio of about 1:1 or higher.

In accordance with still another aspect of the invention, a method of cooking a liquid or solid edible material is provided. The method includes providing a predetermined amount of a liquid or solid edible material in a container, injecting a predetermined amount of water into a heated cavity, forming a quantity of steam in the cavity from substantially all of the water injected into the cavity, and injecting the steam into the liquid or solid edible material contained in the container wherein the quantity of steam is sufficient to cook the predetermined quantity of the liquid or solid edible material. The container interior may be sealed from the environment during the steam injection.

In accordance with a further aspect of the invention, the method of cooking a liquid or solid edible material includes providing a measuring chamber for containing water, supplying water to the measuring chamber so that the measuring chamber contains the predetermined amount of water, and injecting the predetermined amount of water from the water contained in the filled measuring chamber into the heated cavity of the steam generator.

In accordance with a further aspect of the invention, the measuring chamber is filled to capacity during the supplying of water to the measuring chamber and the amount of water in the measuring chamber when filled to capacity is substantially the same as the predetermined amount of water injected into the cavity.

In accordance with a further aspect of the invention, the method of cooking a liquid or solid edible material includes providing a flow control valve that is openable to allow water to flow therethrough and then automatically is closed after said predetermined amount of water has flowed through the flow control valve. Water is supplied to the flow control valve to inject said predetermined amount of water into the heated cavity.

Other advantages and features of the invention will become apparent from the following description and from reference to the drawings.

### BRIEF DESCRIPTION OF THE DRAWINGS

FIG. 1 is a side elevation view of the apparatus in accordance with the present invention;

FIG. 2 is a front elevation view of the apparatus shown in FIG. 1;

FIG. 3 is a schematic view of the apparatus of the present invention;

FIG. 4 is cross-sectional view of the container mounted to the apparatus of FIG. 1 showing the liquid bulk food product and the steam injection wand;

FIG. 5 is an exploded view of the container and wand assembly of FIG. 4;

FIG. 6 is a top exploded view of the wand assembly and container for the apparatus shown in FIG. 1;

FIG. 7 is a cross-sectional view of the container mounted to the apparatus in accordance with a second embodiment of the invention that includes a temperature probe for sensing the temperature of a bulk product in the container;

FIG. 8 is a schematic view of the second embodiment of the apparatus of the present invention that includes the temperature probe illustrated in FIG. 7;

FIG. 9 is a cross-sectional view of the container mounted to the apparatus of FIG. 1 showing the liquid bulk food product and another embodiment of the steam injection wand;

FIG. 10 is an exploded view of the container and wand assembly of FIG. 9;

FIG. 11 is a side elevation view of the apparatus in accordance with a third embodiment of the present invention;

FIG. 12 is a front elevation view of the apparatus shown in FIG. 11;

FIG. 13 is a schematic view of the third embodiment of the apparatus of the present invention;

FIG. 14a is a partial schematic view of the apparatus in accordance with the third embodiment of the present invention illustrating the apparatus in the inactive stage;

FIG. 14b is a partial schematic view of the apparatus in accordance with the third embodiment of the present invention illustrating the apparatus during the measuring chamber filling stage;

FIG. 14c is a partial schematic view of the apparatus in accordance with the third embodiment of the present invention illustrating the apparatus with a filled measuring chamber;

FIG. 14d is a partial schematic view of the apparatus in accordance with the third embodiment of the present invention illustrating the apparatus during the pulse injecting stage; and

FIG. 15 is a schematic view of a fourth embodiment of the apparatus of the present invention.

### DETAILED DESCRIPTION

While this invention is susceptible of embodiment in many different forms, there are shown in the drawings and described in detail herein, several specific embodiments with the understanding that the present disclosure is to be considered as exemplifications of the principles of the invention and is not intended to limit the invention to the embodiments illustrated.

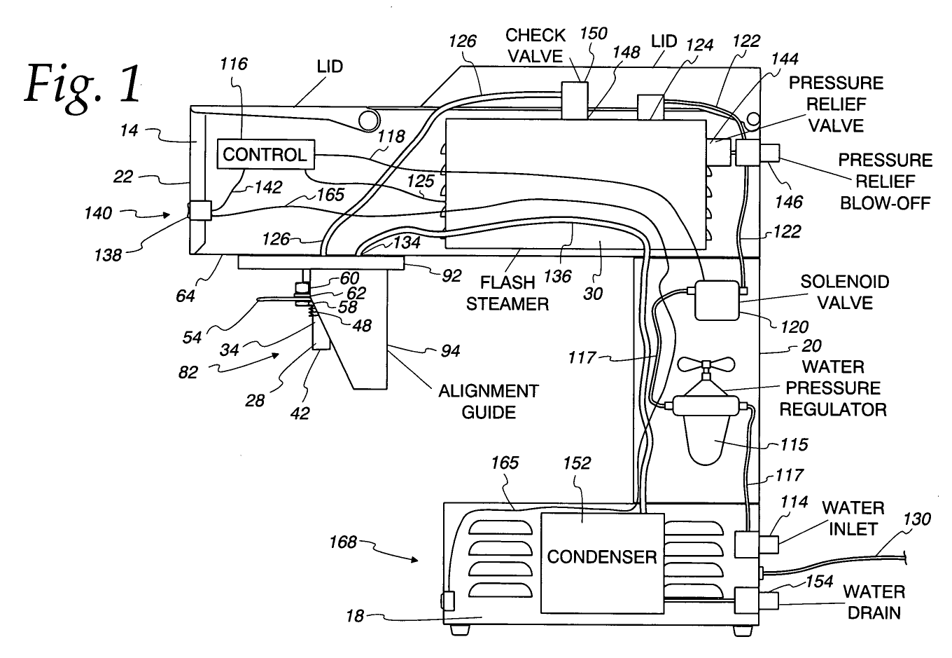

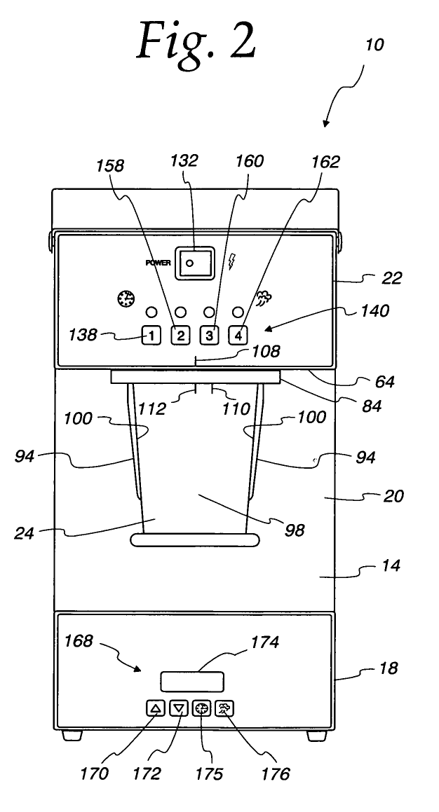

Referring to the FIGS. 1-6, an apparatus 10 for heating a bulk food product 12 by steam injection is illustrated. The present invention provides an efficient low power usage method and apparatus for steam injection heating relatively smaller amounts of bulk food product wherein most of the energy consumed is put into heating the bulk food. In some instances, the heating of bulk food product 12 may be for the purposes of cooking bulk food product 12. In other instances, the heating may be merely to heat a previously cooked bulk food product 12. Apparatus 10 includes a housing 14 with a base 18, a neck 20 and upper housing portion 22. A bulk food product 12 to be heated by apparatus 10 is placed in a cup or container 24 which is removably mounted to apparatus 10. When cup 24 is in the mounted position, a steam injection wand 28 is positioned proximate to bulk food product 12 held in cup 24. Steam formed by a flash steamer 30 is injected into bulk food product 12 to accomplish rapid heating by the injection of steam into bulk food product 12. Any flash steamer 30 can be used in accordance with the invention. Typically, a flash steamer will be comprised of a cavity 32 having a heated surface into which water is injected to rapidly form steam in the heated cavity.

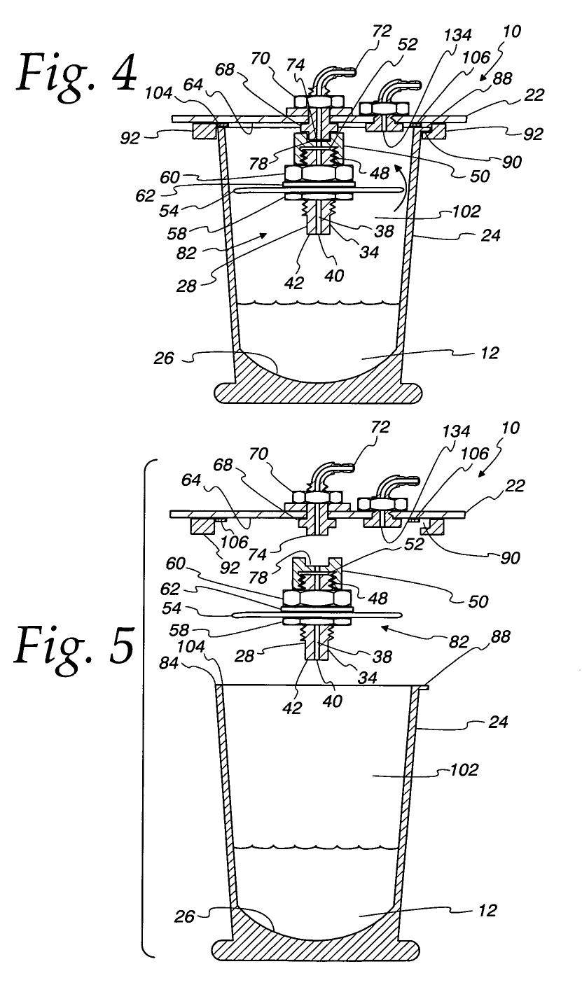

As best seen in FIGS. 4 and 5, wand 28 has a generally cylindrical body 34 with an axially centered steam passageway 38 extending therethrough. Passageway 38 has a steam injection exit port 40 that preferably is located at the bottom or terminal end 42 of wand 28. In the preferred embodiment, terminal end 42 of wand 28 is positioned above the intended level of bulk food product 12 as it is contained in mounted cup 24 prior to thermalization. Typically, passage 38 will have a single steam exit port 40 that preferably directs exiting steam directly downwardly toward bulk food product 12 to thereby maximize the agitation and mixing of bulk food product 12. Wand 28 has a threaded upper portion 48 for mounting a quick release connector 50 at top end 52 of wand 28. Also positioned on wand upper portion 48 is a disc-shaped baffle 54 that is secured in place by nuts 58, 60 and washer 62. Baffle 54 is provided to minimize splattering of bulk food product 12 held in cup 24 as it is injected with steam through wand 28. During steam injection, baffle 54 redirects upwardly splattering bulk food product 12 downward back into the mass of bulk food product 12.

The lower surface 64 of upper housing portion 22 has mounted thereto a quick release connector 68 for cooperation with quick release connector 50 on wand 28. Quick release connector 68 is secured in place by nut 70, and has an inlet end 72 for connection to a source of injected steam from flash steamer 30. Quick release connecter 68 has an outlet end 74 that is inserted into the inlet end 78 of connector 50 to couple together quick release connectors 68 and 50. Typically, the wand assembly 82, which includes wand 28, wand quick release connector 50 and baffle 54, may remain mounted to apparatus 10 except when removed for cleaning.

The rim 84 of cup 24 includes one or more outwardly extending mounting lugs 88 for removably locking within recesses 90 that are provided in an annular skirt 92. Annular skirt 92 may be secured to the bottom surface 64 of upper housing 22 by any suitable means, or may be integrally formed with upper housing 22. When mounting cup 24 to apparatus 10, lugs 88 are first pre-aligned proximate to respective recesses 90, and cup 24 is then rotated to lock lugs 88 within recesses 90. For facilitating pre-alignment of cup 24 during mounting, apparatus 10 includes a generally arcuate-shaped alignment guide 94 that depends downwardly from bottom surface 64 of upper housing 22. When mounting cup 24, a user positions the outer surface of the back side of cup 24 (i.e. the side that is opposite of the front side 98 of cup 24) against the inner surface 100 of alignment guide 94. Cup 24 is then manually moved upwards along inner surface 100 of guide 94 until rim 84 of cup 24 abuts a cup sealing surface, such as lower surface 64 of upper housing 22. Optionally, an annular seal 106 may also be included between rim 84 and surface 64 to more positively seal the interior 102 of cup 24 when cup 24 is mounted to apparatus 10. Seal 106 may be mounted to lower surface 64, top surface 104 of cup rim 84, or may be a separate removable piece.

Alignment reference markings 108 and 110 are provided in order to allow a user to readily accomplish pre-alignment of lugs 88 relative to their respective cooperating recesses 90. When alignment markings 108 and 110 are vertically aligned, each lug 88 of cup 24 is in a pre-aligned positioned proximate to the opening of a cooperating recess 90. With cup rim 84 abutting the sealing surface provided by lower surface 64 of housing 22, cup 24 is manually rotated to lock lugs 88 within recesses 90 to thereby removably secure cup 24 to apparatus 10. A reference marking 112 may also be provided on cup 24 that vertically aligns with marking 108 to indicate that cup 24 has been properly moved to the locked position on apparatus 10. To release and remove cup 24 from apparatus 10, cup 24 is rotated in the opposite direction to return lugs 88 to an unlocked position relative to recesses 90. Cup 24 is then moved downward and withdrawn from apparatus 10. In this manner, apparatus 10 is provided with means allowing for convenient and relatively rapid mounting and releasing of cup 24, which is especially advantageous for use in a QSR where multiple food orders need to be heated in order to fill numerous food orders received during peak ordering times.

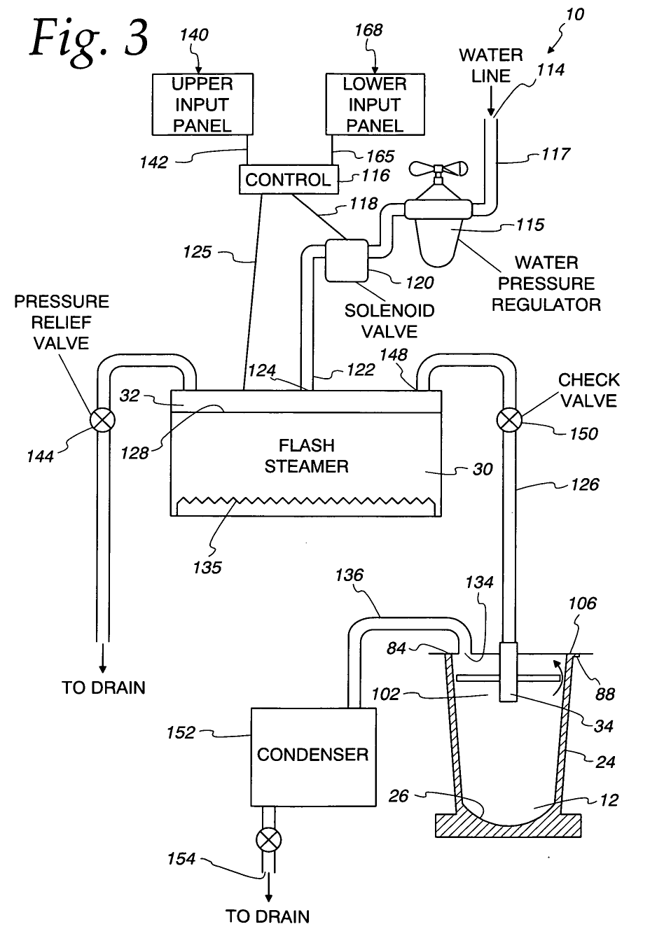

FIG. 3 is a schematic illustration of the system of apparatus 10. Apparatus 10 includes a water inlet line 114 that is connected to a source of water, such as a water line of the Quick Service Restaurant in which apparatus 10 is used. A water pressure regulator 115 is provided along conduit 117 to maintain inlet water pressure for apparatus 10 to a desired level for flash steamer 30, which typically will be in the range of about 20 psi to about 60 psi. A liquid water injector, such as a solenoid valve 120, is openable to allow water to flow from water inlet line 114 through solenoid valve 120 to water conduit 122 for injection into flash steamer 30. Flash steamer 30 has an inlet 124 to receive a quantity of liquid water injected into flash steamer 30. The injected water is directed against a heated cavity surface 128 in the heated cavity 32 of flash steamer device 30 to cause rapid conversion into steam of substantially all of the injected quantity of water.

In the operation of apparatus 10, solenoid valve 120 opens momentarily to cause a pulse of a predetermined quantity of water to be injected into flash steamer 30. To accomplish this, a control 116 is connected to solenoid valve 120 by cable 118. Control 116 provides a signal to cause solenoid valve 120 to open for a relatively short period of time, and then cause solenoid valve 120 to return to its normally closed position after the predetermined quantity of water has been injected. Control 116 includes a micro-processor controller having suitable software and electronically stored programmed data necessary to provide injected water pulses in predetermined amounts in accordance with the invention.

In one preferred mode of operation, the predetermined amount of injected water is that which is sufficient to produce an amount of steam at flash steamer 30 that is sufficient to cook a predetermined single serving size of a particular type of bulk food product 12 placed in cup 24. When the predetermined quantity of water is injected into heated cavity 32, flash steamer 30 converts substantially all of the predetermined quantity of injected water into steam. Thereafter, substantially all of the steam produced at flash steamer 30 exits flash steamer 30 through conduit 126 traveling to wand 28 and out exit port 40 to be injected into bulk food product 12 to cause injection steam cooking. The steam traveling through wand 28 is of a sufficient velocity to cause mixing of bulk food product 12 in container 24. Typically, the velocity of the steam is in the range of from about 250 feet per second to about 800 feet per second, and more typically from about 400 feet per second to about 700 feet per second. An exemplary period of duration of the peak steam output through wand 28 is in the range of about 3 seconds to about 4 seconds. In order to enhance mixing of the pressurized bulk food product 12 and to reduce trapping of bulk food product 12 on the bottom of cup 24 during pressurization, cup 24 is preferably provided with a concave interior bottom surface 26, such as for example an inverted generally dome shape. Thus, as bulk food product 12 adjacent the concave interior bottom surface 26 is pressurized by steam from wand 28, bulk food product 12 may be caused to move along the upward slope of concave interior bottom surface 26 and into the remainder of the mass of bulk food product 12.

Any type of food that can be heated and/or cooked with steam can be used with the device and method of the present invention. Customary types of bulk food product 12 that can be cooked and/or heated typically include raw eggs, such as fresh, shelled eggs, or pasteurized whole egg (PWE) to make scrambled eggs, rice, noodles, pasta, stew, soup, for example. A single serving amount of bulk food product 12 can be any desired size. Typically, the single serving size is selected to be the size of the minimum order for each type of bulk food 12 served in a particular quick service restaurant. By way of example, for a scrambled eggs order, the typical single serving size may be that which is produced from 1 or 2 uncooked raw eggs (e.g., fresh, shelled eggs or an equivalent amount of PWE), most typically 2 eggs. To enable apparatus 10 to cook more than one type of bulk food product 12, control 116 may be programmed to be able to provide a plurality of different predetermined injected water pulse sizes. More specifically, control 116 is programmed to allow a user to select a pulse size from among the different stored pulse sizes and then inject the selected pulse size into flash steamer 30. Each of the stored predetermined pulse sizes is a particular predetermined amount of injected water that is most advantageous for cooking a single serving size of a particular type of bulk food product 12 by apparatus 10. Still more specifically, a selectable predetermined pulse size is the predetermined amount of injected water that, when injected into heated cavity 32 and substantially all of the injected water is converted into steam, is sufficient to produce a quantity of steam that is sufficient for cooking a single size serving of the particular selected type of bulk food 12.

Apparatus 10 may also be used to cook bulk food product 12 in predetermined amounts that are integer multiples, such as 2, 3, 4, or greater, of the single serving size for a particular type of bulk food product 12, which may be, for example, 2, 3, 4 or more fresh, shelled eggs or PWE equivalent or any other type of food as desired. As discussed in greater detail below in connection with cooking scrambled eggs, the preferred method of cooking integer multiple serving sizes is by the injection of a number of successive multiple water pulses, the number of the successive pulses being equal to the integer number of serving sizes being cooked. The amount of liquid water injected in each pulse is the predetermined amount sufficient to cook a single serving of the bulk food product type. For example, to cook a double serving size, two successive water pulses are injected with each pulse being the predetermined amount of injected water to cook a single serving size.

In order to more fully understand the apparatus and method of the present invention, a more detailed description of the exemplary cooking of a single serving size of scrambled eggs, which may be two eggs, for example, is provided. To begin, two eggs are broken and their contents of white and yolk are placed in cup 24 to form a liquid mass of bulk food product 12. Alternatively, rather than using raw eggs to produce a single serving of scrambled eggs, an equivalent predetermined quantity of pasteurized whole egg (PWE) may be used. PWE provides the advantage of being easier and quicker to dispense into cup 24, when compared to raw eggs that must first be cracked and carefully deposited into cup 24 to avoid spillage. To cook an equivalent two egg single serving size of scrambled eggs from PWE, a predetermined amount for producing a single serving size is placed in cup 24, which may be in the amount of about 4 fluid ounces.

With power cord 130 connected to a source of electrical power and apparatus 10 turned on at power switch 132, cup 24 containing the single serving of bulk egg food product 12 is sealingly mounted to apparatus 10. Optionally, apparatus 10 may also include a safety interlock feature to prevent operation of apparatus 10 unless cup 24 is properly mounted to apparatus 10. For example, sensors (not shown) may be included to signal control 116 that all of lugs 88 are properly positioned in recesses 90 to indicate cup 24 is properly mounted. If proper mounting is not sensed, control 116 prevents operation of apparatus 10 until proper mounting of cup 24 is accomplished.

To initiate a cooking cycle of a single serving of scrambled eggs, a user selectively depresses button 138 of upper input panel 140. Button 138 may be labeled as number “1,” or may have more specific indicia, such as “cook single scrambled egg serving”. The user's selection of button 138 is communicated over cables 142 to control 116. After receiving this signal, control 116 opens solenoid valve 120 to cause the injection into flash steamer 30 a predetermined quantity of water that is sufficient, when substantially fully converted into steam, to cook a predetermined single size serving of scrambled eggs, i.e. two eggs. To accomplish cooking of a single serving size of scrambled eggs in the amount of two eggs, control 116 opens solenoid valve 120 for a short period of time, typically, such as about 0.95 seconds to thereby inject a predetermined quantity of water of about 1.4 oz. As described in more detail below, by injecting water in this amount and manner, flash steamer 30 produces a supply of steam providing a cooking cycle wherein the steam is injected through wand 28 for about 10 seconds. The ten second cooking cycle is the preferred amount of time sufficient to produce a single serving size of scrambled eggs in the amount of two eggs.

It is noted that by some accounts scrambled eggs produced by steam injection may be considered to have a somewhat washed out taste. Unexpectedly, it was found that by using the PWE as the bulk liquid egg to produce scrambled eggs in accordance with the present invention, and by increasing the solid content of the PWE to be in the range of 23% to about 25% by weight, the taste of the resulting scrambled eggs was considered to be superior. Indeed the taste was surprisingly found to be even better than scrambled eggs made traditionally by cooking on a grill.

It is further noted that while in the preferred embodiment the predetermined amount of the liquid water injection pulse is controlled by the period of time solenoid valve 120 is opened, those skilled in the art will recognize that this may be alternatively accomplished by other suitable means. For example, apparatus 10 may be provided with a flow meter device (not shown) that measures the amount of injected water of a pulse and sends a signal to control 116 when the desired predetermined quantity of water has been provided for injection into flash steamer 30, and thereafter control 116 closes solenoid valve 120.

In addition to controlling the amount of water injected into flash steamer 30, control 116 also may control and monitor the operation of flash steamer 30. Connected to flash steamer 30 by cable 125, control 116 may be programmed to control the preheating of steam generator surface 128, maintain steam generator surface 128 at a predetermined temperature, and otherwise monitor the operation of flash steamer 30. Typically, the temperature of steam generator surface 128 will be maintained at about 400° F. by control 116 powering on and off the exemplary heating means of electrical heating element 135. The typical temperature of the steam generated by flash steamer 30 is about 230° F. or more.

A pressure relief valve 144 is provided for flash steamer 30 to prevent excessive build up within heated cavity 32 of flash steamer 30. For safety and regulatory reasons, pressure relief valve 144 typically opens to vent cavity 32 whenever the pressure in cavity 32 exceeds a pre-selected pressure amount, for example about 15 psig, or lower. Vented steam is released through a blow-off conduit 146 that directs the vented steam to a safe location away from the operator of apparatus 10.

Upon injection of the predetermined quantity of liquid water, the injected water is rapidly converted into steam which thereafter rapidly pressurizes cavity 32. The pressurized steam flows out from cavity outlet 148 and continues downstream along steam conduit 126. A one way check valve 150 is preferably included to prevent the steam from flowing back towards flash steamer 30. The supply of steam travels through conduit 126 and enters passageway 38 of wand 28. Thereafter, the pressurized steam downwardly exits exit port 40 of wand 28 to be injected into bulk food product 12. As the steam is injected into the substantially sealed cup interior 102, the pressure of cup interior 102 is rapidly increased. The injection of steam vigorously agitates liquid bulk egg product 12. As noted previously, during the steam injection cycle, bulk food product 12 that is forced upward by the pressurized steam contacts baffle 54 and is redirected by baffle 54 back downward into the cooking food mass being formed into scrambled eggs. The agitation by the steam injection causes rapid mixing and relatively uniform heat transfer to all portions of the turbulent liquid bulk food product 12. Any excess steam that is not used for cooking bulk food product 12 is vented from cup interior 102 at outlet vent 134 through conduit 136 leading to a condenser 152. At condenser 152, the excess steam is condensed into liquid water that drains at a drain 154.

It is noted that once the predetermined quantity of water is injected through solenoid valve 120 and into flash steamer 30, no further downstream control of the cooking cycle of apparatus 10 is required. The operation of the cooking cycle self terminates when substantially all of the steam produced at flash steamer 30 is depleted. This occurs when the steam has flowed through conduit 126 and wand 28 to be injected to cook the bulk food product 12, with any excess steam exiting cup 24 at outlet vent 134. Thereafter, the pressure of cup interior 102 quickly returns to atmospheric pressure. The length of the steam injection cycle, that period of time of the cooking cycle during which steam is injected into bulk food product 12, is dependent upon a number of factors. These factors include the amount and the pressure of steam generated by the predetermined injected amount of water, the length of the path the steam must travel from flash steamer outlet 148 to exit port 40 in wand 28, the inside diameter of steam conduit 126, the sum of the cross sectional areas of exit port 40 or exit ports 40, if more than one, and the inside diameter of wand 28. Additionally, the duration of the steam injection cycle may be dependent upon the effective cross sectional area of the opening through quick release connectors 50, 68 and check valve 150 and flash steamer outlet 148 if their passageways choke the flow of steam therethrough, i.e., their inside diameter is appreciably less than the remainder of the flow path for the steam.

For example, a steam injection cycle of about 10 seconds can be accomplished by injecting a predetermined quantity of water of about 40 milliliters creating a steam cavity pressure of about 7 psig, providing steam conduit 126 with an inside diameter of about ¼ inch and a steam pathway length i.e. the combined length of conduit 126 and wand 28 of about 16 inches, with an average inside diameter of the steam passageway 38 of wand 28 being about ⅜ inch, and a total cross sectional area opening size of ¼ sq. in. for exit port 40. Typically the length of conduit will be in the range of about 8 inches to about 16 inches.

It is to be understood that one of the advantages of apparatus 10 is that once the steam exits flash steamer 30, no downstream control of the steam is required. The steam injection cycle into bulk food product 12 simply ends when the formed steam is depleted. However, it is also to be understood that the injection of steam into bulk food product 12 could be terminated by other means. For example, apparatus 10 may include a valve (not shown) located between flash steamer 30 and wand 28, such as along conduit 126. Such valve would be closed by control 116 after a desired period of time of steam injection cooking, for example 10 seconds. Closing this valve would terminate further any downstream movement of steam past the valve toward bulk food product 12, regardless of whether all the steam produced in heated cavity 32 has been depleted.

As discussed previously, apparatus 10 can also readily be used to cook integer multiples of a single size food serving. This includes cooking a predetermined scrambled egg amount that is two, three, four or more times the size of a desired single serving size of scrambled eggs. For example, if the predetermined single serving size is two eggs, to cook a double size serving of scrambled eggs, four uncooked shelled eggs (or the PWE equivalent) is placed in cup 24. Cup 24 is thereafter mounted to apparatus 10. The user then activates the cooking cycle for the double serving size of scrambled eggs by pushing button 158. Button 158 may have indicia that read, for example, “2” or “cook two servings scrambled eggs.” Selection of button 158 generates a signal to control 116 that a double size serving of scrambled eggs is to be cooked. Upon receiving such signal, control 116 causes water to be injected into flash steamer 30, preferably as two separate and substantially equal water pulses. The amount of each of the injected water pulses is the predetermined amount of water injected for cooking a single size serving of scrambled eggs. Thus, the total predetermined amount of the two injected pulses is sufficient to cook a double size serving of scrambled eggs.

As is the case with cooking accomplished by a single pulse of injected water, substantially all of the injected water from the two sequentially injected pulses is converted into steam by flash steamer 30, and in turn, substantially all of the formed steam is injected into the bulk eggs 12. The injection of two pulses of water is accomplished by control 116 opening solenoid valve 120 to inject the first pulse of water, closing solenoid valve 120, and after a predetermined period of time, reopening solenoid valve 120 to inject the second pulse of water. The second pulse of liquid water is preferably injected in the latter half of the steam injection cooking cycle produced by the injection of the first pulse of injected water. This is done so as to accomplish one continuous cooking cycle for bulk food product 12. More specifically, the timing of the second pulse of injected liquid water is such that the steam formed from the second pulse travels downstream to reach the bulk food product 12 before the termination of the steam injection cycle resulting from the first injected pulse of liquid water. For example, when cooking a double serving of scrambled eggs in a predetermined amount of four eggs, or the PWE equivalent thereof, a first pulse of liquid water of about 1.4 ounces is injected into flash steamer 30 by opening solenoid valve 120 for about 0.95 seconds. The first pulse of injected water produces steam that is sufficient to cause a steam injection cycle of about ten seconds. At a time before this steam injection cycle ends, most preferably after about 8 seconds from initiation of the first injected water pulse by control 116, the second pulse of liquid water injected by solenoid valve 120 is initiated by control 116. The amount and duration of the second pulse preferably is the same as the first injected pulse, i.e., approximately about 0.95 seconds to inject about 1.4 ounces of liquid water. By timing the pulses in this manner, the steam generated from this second pulse of injected water will reach wand exit ports 40 for injection into bulk food product 12 before the steam injection cycle generated from the first pulse has ended. Thus, the two spaced apart first and second injected water pulses provide the preferred one continuous cooking cycle. Preferably, the total period of the steam injection cycle into bulk food product 12 resulting from steam generated from both the first and second pulses of injected liquid water is fifteen seconds, which is the preferred duration of the cooking cycle used to cook the double serving of scrambled eggs.

Providing two spaced apart approximately equal water pulses as described above results in the cooking conditions for a double serving that are generally similar to the conditions occurring when cooking a single serving of eggs with a single injected pulse of water. This is because the steam created by the second pulse of injected water does not reach cup interior 102 until the majority of the steam created from the first pulse has already been depleted by the cooking of bulk food product 12. Thus, the pressure, temperature, and amount of agitation of bulk food product 12 are similar whether cooking a single or double serving size. The use of multiple pulses also reduces the stresses to which apparatus 10 is subjected and also enhances safe operation of apparatus 10. This is because the additional steam resulting from the second pulse is formed after a majority of the steam produced by the first injected pulse of water has already exited from the heated cavity 32. This limits the amount of steam that is present in cavity 32 at any given point in time to thereby limit the pressures and stresses to which apparatus 10 is subjected.

Another advantage achieved in heating by using substantially equal water pulse amounts, is that calibration of the water injection cycle through solenoid valve 120 is simplified. This is because apparatus 10 needs only to be calibrated to deliver the predetermined amount of water to cook a single serving of a type of bulk food product, and this same predetermined pulse amount is repeated as a second, third, or fourth pulse to respectively cook a double size, triple size or quadruple size serving.

While the preferred method is to cook a double serving size with two separate pulses, it is to be understood that the invention could alternatively be practiced by using a single larger pulse. Such larger single pulse being an amount that is double the predetermined quantity that is used to cook a single serving size of scrambled eggs. However, when using a larger single pulse to cook a quantity of bulk food product 12 that is an integer multiple of a single serving, the larger single pulse may affect cooking results, subject cooking apparatus 10 to additional stresses, and/or cause the pressure relief valve 144 of flash steamer 30 to open. For example, the single pulse injection of an amount of water that is quadruple that for a single serving of scrambled eggs, for example, by opening solenoid valve 120 once for a period of about 3.8 seconds (4 times 0.95 seconds) would rapidly produce a relatively large quantity of steam in heated cavity 32. This increased amount of steam is more likely to cause pressure relief valve 144 to open as the result of the pressure in cavity 32 exceeding the pre-selected release pressure. This would be undesirable since it would cause the venting of steam for safety reasons, rather than utilizing the steam for injection into bulk food product 12 to accomplish heating.

Apparatus 10 may be likewise used to cook servings of various types of food as desired other than scrambled eggs. These other types of food, for example, may include rice, noodles, pasta, stew, and soup. Such other types of bulk food product also may be cooked in a desired predetermined single size serving amount, or an integer multiple thereof. For example, a desired predetermined single serving size of noodles of from about 4 ounces to about 8 ounces by weight may be cooked by placing a single serving size of noodles in cup 24 and mounting to apparatus 10 as previously described. A food type selection input button (not shown) is included in upper control panel 140 to allow the user to select noodles as the type of food to be cooked. Thereafter, to cook a single size serving of noodles, the user selects noodle cooking and then depresses a control button 138 marked “1”. Control 116, after receiving a signal that button 138 has been depressed to cook a single size serving of noodles, opens solenoid valve 120 to cause a pulse of water of a programmed predetermined amount to be injected into flash steamer 30. In this instance, the programmed predetermined amount is that which is sufficient, when substantially fully converted to steam at flash steamer 30, and thereafter substantially all of the steam formed is injected in the bulk noodles for steam injection cooking, is sufficient to cook the single serving of bulk noodle food product 12. Also, in a similar manner described above for cooking a double serving of scrambled eggs, to cook a double serving size of noodles in cup 24, control 116 opens solenoid valve 120 to inject into cavity 32 a first pulse of water in the predetermined quantity for cooking a single serving of noodles and, after a short period of time, injects a second pulse of water. The amount of the second pulse of injected water is also in the predetermined quantity sufficient for cooking a single serving of noodles. The second pulse of injected water is preferably likewise timed to take place in the latter half of the steam injection cycle provided by the first pulse of injected water, so that one continuous steam injection cycle is provided to cook the double serving size of noodle bulk food product 12.

In cooking predetermined quantities of bulk food product 12 in the amount of integer multiples greater than two, a similar operation as described above is followed. For cooking serving sizes of a bulk food 12 quantity that is 3 or 4 times that of a single serving, 3 or 4 pulses, respectively, of injected water are used. To select the serving size to be cooked, and to initiate its cooking, the user depresses input button 160 for a triple size serving or input button 162 for a quadruple serving size. Control 116 times the injection of the third and fourth pulses of liquid water into flash steamer 30 so as to occur before termination of the second and third steam injection cycles, respectively. Typically, the third pulse of injected water is initiated during the latter half of the second steam injection cycle, and the fourth pulse is initiated in the latter half of the third steam injection cycle. Thus, whether one, two, three, or four serving sizes are cooked by apparatus 10, a continuous steam injection cooking cycle is provided for bulk food product 12. It is noted that to accommodate larger volumes of bulk food product 12, the diameter of cup 24 may be increased so as to allow terminal end 42 of wand 48 to be in a position that is above the surface level of such larger volumes of bulk food product 12 to be thermalized.

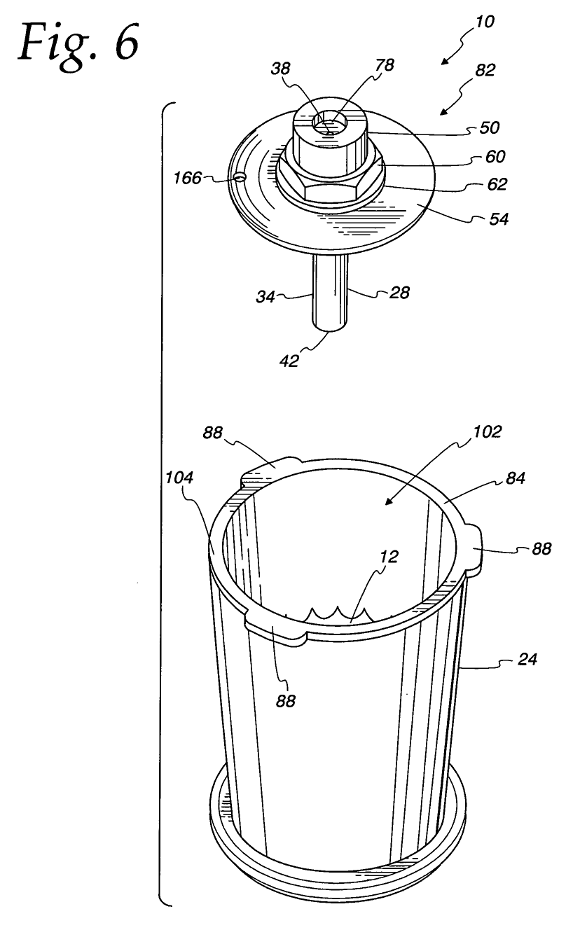

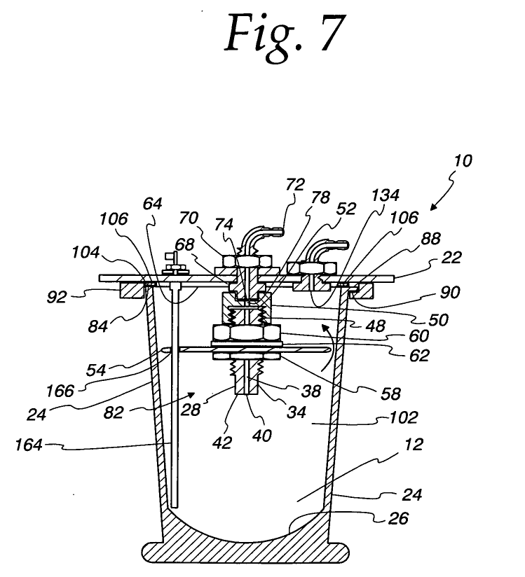

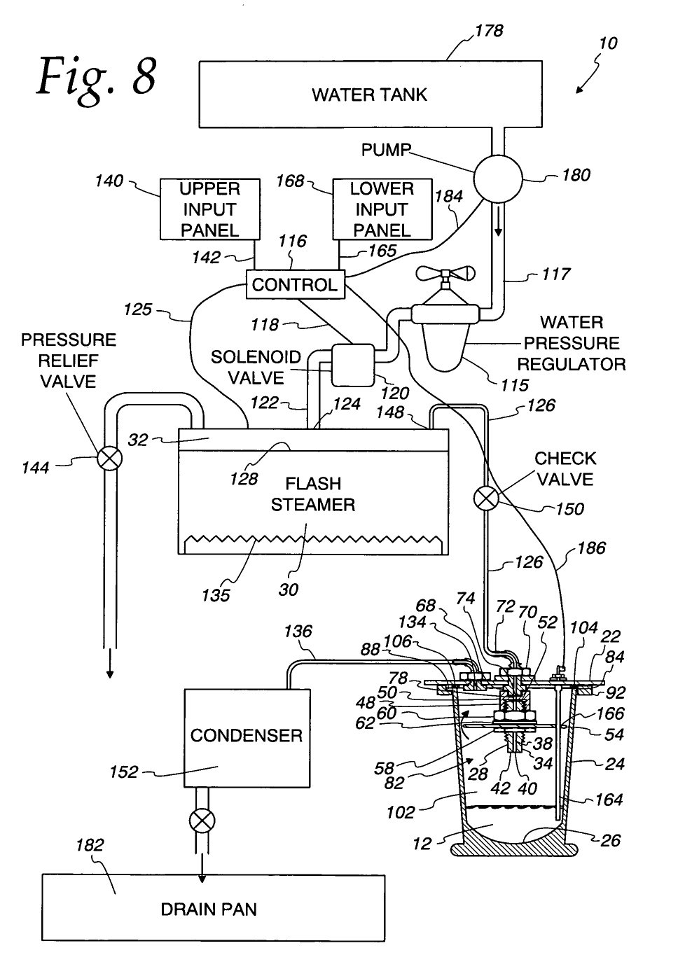

FIGS. 7 and 8 illustrate a second embodiment of the invention which may be used for heating bulk food product 12 by injecting steam formed from a plurality of relatively rapid, successive injections of pulses of water into flash steamer 30. Heating continues until a pre-selected temperature is achieved for the bulk food product 12. This embodiment of the invention includes a temperature probe 164 mounted to apparatus 10 to extend downward from lower surface 64 of housing upper portion 22. When cup 24 containing bulk food product 12 is mounted to apparatus 10, temperature probe 164 is inserted through a slot or hole 166 in baffle 54 and is immersed in bulk food product 12. Hole 166 is shown in FIG. 6 for purposes of illustration of a modification of baffle 54 that can be used with the embodiment of apparatus 10 shown in FIGS. 7 and 8. In embodiments of apparatus 10 not having temperature probe 164, baffle 54 preferably would not include a slot or hole 166. Preferably, temperature probe 164 is spaced a distance away from wand 28 and its exit ports 40. This allows the temperature sensed by temperature probe 164 to more accurately reflect the temperature of bulk food product 12. If placed too close to exit port 40, the temperature sensed at probe 164 is more likely to reflect the temperature of the injected steam, rather than that of the bulk food product 12. The sensed temperature is communicated from temperature probe 164 to control 116 through a cable 186. Other suitable means for sensing the temperature of bulk food product 12 known to those skilled in the art alternatively may be used. For example, apparatus 10 may sense temperature through infrared sensing or with a thermometer.

Before initiating heating or cooking in this mode of operation, a user enters a pre-selected temperature at lower input panel 168. This is accomplished by depressing temperature “up” button 170 and/or temperature “down” button 172 until the desired final heating temperature for bulk food product 12 is displayed at display 174. Thereafter, the start steaming button 176 is depressed to begin the heating process.

It is noted that another difference in the operation of the second embodiment is that bulk food product 12 being heated need not be in the amount of a single serving size or integer multiples thereof. The amount of bulk food product 12 may be an amount less than a single serving, or any other greater amount that can be effectively contained within the cooking capacity of cup 24. Another important difference is that the quantity of pulses of water injected into flash steamer 30 is not based on a predetermined amount of liquid water that is sufficient, when converted to steam, to cook a single serving size of bulk food product 12. Instead, the amount of the pulse of injected water is relatively small. Such smaller pulses are repeatedly injected into flash steamer 30 to continuously produce pulses of steam that are injected into bulk food product 12 to incrementally raise the temperature of bulk food product 12. When the pre-selected temperature is achieved in bulk food product 12, and is sensed by probe 164, control 116 ceases injecting water pulses through valve 120 into heated cavity 32.

The use of multiple relatively smaller pulses of injected water not only provides flexibility in heating a wider range of bulk food amounts and types, but also allows a wider range and more precise control of heating temperatures to be achieved. This is because each of the smaller pulses accomplishes a smaller amount of heating when compared to the mode of operation wherein a relatively large single pulse or small number of pulses of water are used to cook an entire single bulk food serving. The use of such larger pulses can make it more difficult to precisely cook or heat food amounts less than a single serving size, or non-integer multiples of a single serving, i.e. 1½, 2¾ single servings, etc. Also, each of the relatively larger pulses provides a heating capacity that is too great to allow accurately heating to a specific selected temperature.

It is noted that with the injection of the smaller pulses of water, flash steamer 30 still produces steam in the same manner as described above in connection with the larger pulses. The smaller amount of water of each pulse, however, results in the production of a smaller amount of steam by flash steamer 30. Thus, the plurality of relatively smaller rapid water pulses injected at flash steamer 30 provide a plurality of sequential relatively smaller steam injection cycles during which steam is injected into bulk food product 12 to cook and/or raise its temperature. When temperature probe 164 sends a sensed temperature to control 116 that is at least the temperature pre-selected by the user, control 116 ceases injecting additional pulses of water through solenoid valve 120. At this time display 174 displays a message such as ‘ready’ or ‘cooking completed’ and/or provides an audible signal indicating that the cooking or heating cycle has been completed. Thereafter, cup 24 is removed from apparatus 10 and bulk food product 12, now heated to the user selected temperature, is then removed from cup 24 for consumption.

Optionally, the second embodiment of the invention may include various pre-programmed heating functions for which apparatus 10 is commonly used. For example, control 116 may be pre-programmed to be capable of automatically performing a function such as reheating noodles or cooking eggs. Control 116 may include stored information, including an advantageous “heat to” temperature to accomplish a particular function, and these various functions can be initiated by selecting the appropriate button provided for that purpose (not shown) located at upper or lower input panels 140, 168. Pre-programming such functions is advantageous for ease of operation, and does not require the user to remember, or look up, the particular optimal “heat to temperature” to perform a specific heating or cooking function for a particular type of bulk food product 12.

Lower input panel 168, communicating with control 116 through cable 165, optionally may also include inputs to select a timed heating or cooking cycle. The particular desired period of heating to be performed is entered by the user at lower input panel 168. For example, button 175 is depressed to select the timed heating mode. Up and down buttons 170, 172 are then used to change display 174 to enter the desired heating time. After the desired cooking time is displayed, the start steaming button 176 is depressed to initiate steaming. Once a timed heating operation is initiated, control 116 begins and continues the injection of the smaller pulses of water into flash steamer 30. The steam produced by the pulses is injected into bulk food product 12 through wand 28 until the selected time period ends, for example after 45 seconds. Typically, as in heating the bulk food product 12 to a pre-selected temperature, the smaller pulsed cycles will also take place within a relatively small interval of time therebetween, for example, such as about every 0.1 to 1.0 seconds or more.

Another embodiment of the invention is illustrated in FIG. 8. As shown therein, apparatus 10 is modified to include a water tank or reservoir 178, a pump 180 and a removable drain pan 182. Such modification allows apparatus 10 to be self contained and operate remotely of connection to water inlet 114. Thus, with this modification, apparatus 10 becomes portable and is more suitable for home use wherein apparatus 10 can be moved to any convenient location on a kitchen countertop or other suitable support surface. In the modification of apparatus 10 illustrated in FIG. 8, water tank 178 holds a refillable supply of water. Operation of water pump 180 is controlled by control 116 through cable 184 and pressurizes the water pumped from water tank 178 to flash steamer 30. Typically, the water pressurized by pump 180 is from about 20 psi to about 60 psi or as otherwise needed for the flash steamer being utilized. Downstream of water pump 180 the operation of the system of apparatus 10 is the same as previously described above, except that the self-contained apparatus 10 also includes removable drain pan 182 for collecting discharge water from condenser 152.

The embodiment illustrated in FIG. 8 typically will use the relatively smaller injected water pulses as previously described above in connection with the operation of heating a bulk food product 12 to a pre-selected temperature entered by the user at lower panel 168. However, it is to be understood that this embodiment may also be programmed to accomplish other previously described modes of operation. For example, cooking a single size serving of bulk food product 12 by injection of a single pulse of water of a predetermined quantity into flash steamer 30. As previously described, such predetermined amount of injected water being an amount that when substantially all the injected water is converted to steam, it is sufficient to produce a quantity of steam that is sufficient to cook a predetermined single serving size of bulk food product 12. Also, multiple injections of a predetermined amount of water could be used with the embodiment of FIG. 8 to cook integer multiples of a single size serving, as also previously described.

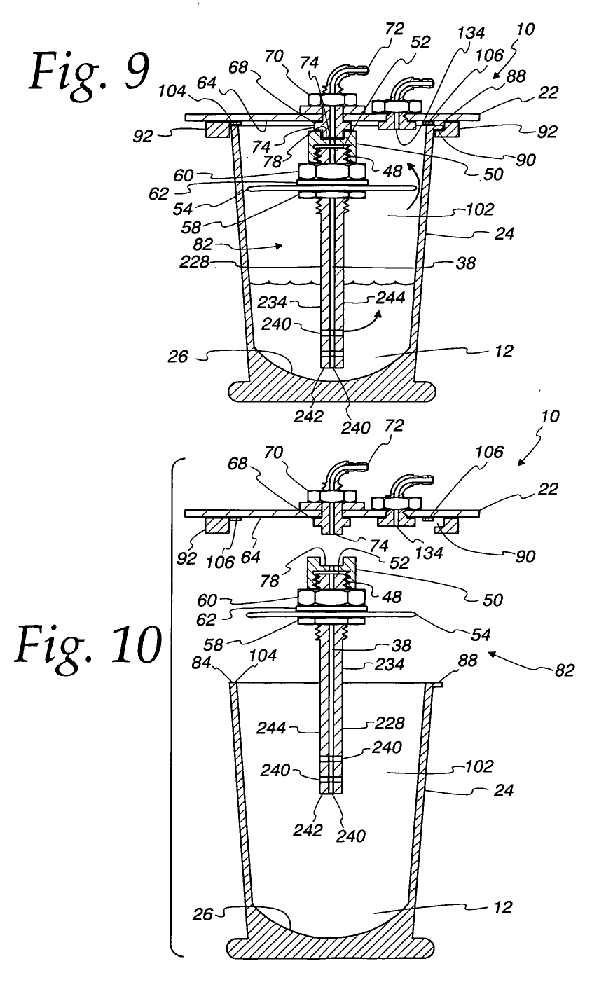

As shown in FIGS. 9 and 10, another embodiment of the invention has a wand 228 that has a body 234 that is of an extended length. Unlike shorter cropped off wand 28 shown in FIGS. 1-8, longer wand 228 extends to be immersed into bulk food product 12 contained in cup 24 when mounted to apparatus 10 for thermalization. The lower portion 244 of longer wand 228 preferably includes multiple steam injection exit ports 240 leading from axially centered steam passageway 38. Preferably one exit port 240 is located at bottom or terminal end 242 of longer wand 228. Additional exit ports 240, in fluid communication with steam passageway 38, preferably are located in a vertically and circumferentially spaced arrangement about lower portion 244 of wand 228. The additional exit ports 240 are also located in a position so that the additional exit ports 240 are immersed in bulk food product 12. It is noted that wand 28, having a shorter length than the longer wand 228, offers an advantage in that wand 28 generally will be easier to clean and be less prone to fouling since wand 28 is not normally immersed into bulk food product 12 as is the case for longer wand 228.

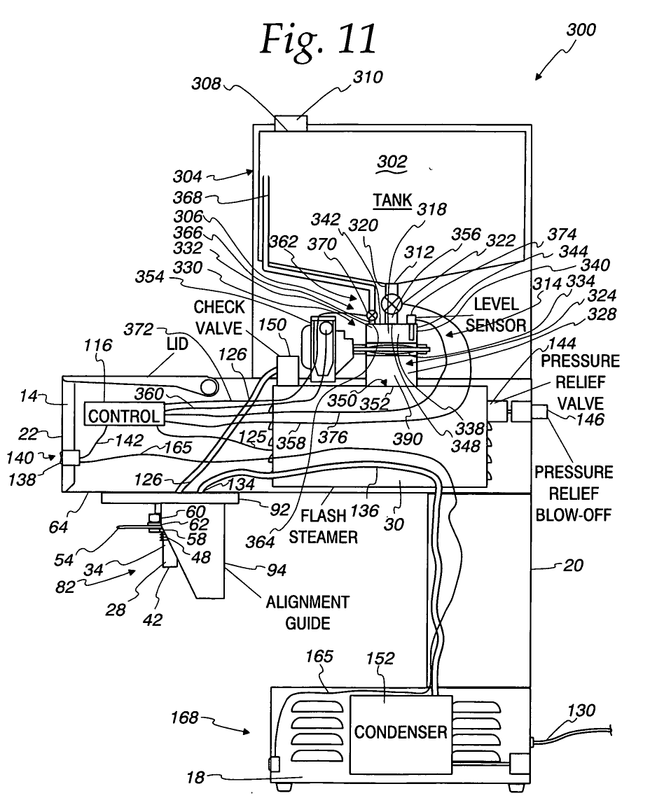

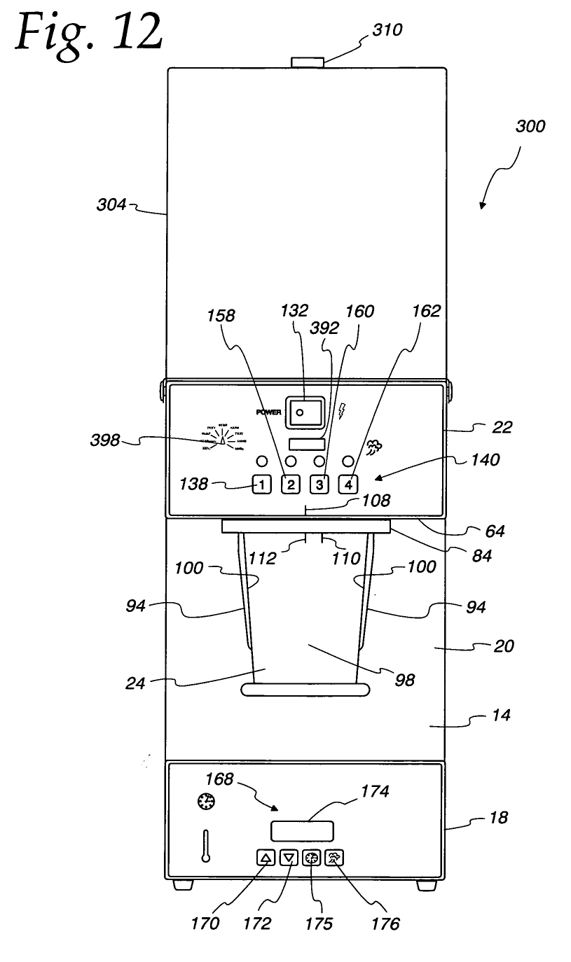

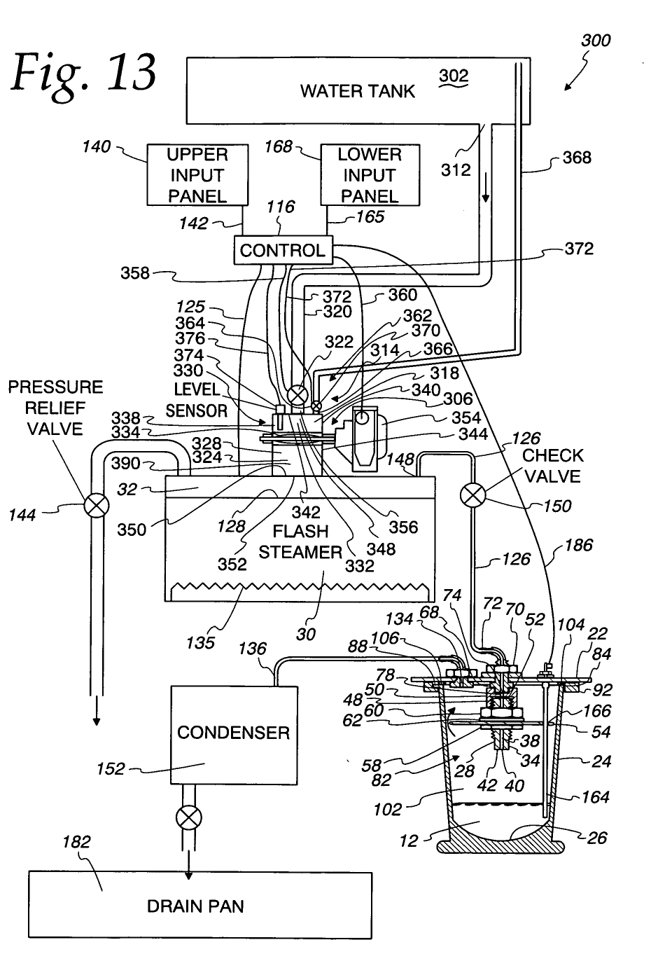

Another embodiment of the present invention is illustrated as apparatus 300 shown in FIGS. 11-14. Apparatus 300 like the embodiment of the invention shown in FIG. 8 also has a water tank 302 to allow apparatus 300 to be self contained and portable. Thus, apparatus 300 is particularly suitable for home use applications. Unlike the embodiment shown in FIG. 8, apparatus 300 does not include pump 180 and has a water injector that is different than solenoid valve 120 shown in FIG. 8. The liquid water injector 306 of apparatus 300 includes a water measuring chamber 318 which is filled from water tank 302 to contain a measured amount of water that is then injected as a pulse of liquid water into flash steamer 30. In the same manner as with the previously described embodiments of the present invention, the injected pulses of water are converted to steam for steam injection heating the contents placed in container 24. Before describing apparatus 300 and its operation for heating, it is noted that the term heating is meant to include the cooking as well as the heating or thermalizing of a liquid or solid edible material.

Apparatus 300 has a vertically extended housing section 304 for accommodating and enclosing water tank 302. Water tank 302 includes a filling inlet 308 for filling water tank 302 as required. Inlet 308 may be sealed by any suitable means, such as removable closure 310 or a housing lid (not shown) provided at the top of housing section 304. A water tank outlet 312 is provided at the bottom of water tank 302. Water tank 302 is elevated relative to flash steamer 30 to allow water contained in water tank 302 to flow by the force of gravity through outlet 312 and towards flash steamer 30.

Positioned along the flow path 314 between water tank 302 and flash steamer 30 is liquid water injector 306 that includes a measuring chamber 318. An upper conduit 320 provides a water passageway from water tank 302 into measuring chamber 318. Positioned along conduit 320 is a measuring chamber inlet valve 322 that is openable to allow the flow of water from water tank 302 when filling water chamber 318. A water passageway 324 provides an exit flow path 328 from measuring chamber 318 to flash steamer 30. As discussed later in greater detail, measuring chamber 318 receives water from water tank 302 to accumulate a measured amount of water that is thereafter released as an injected measured pulse of water to flash steamer 30.

While measuring chamber 318 may be independently housed in a separate component, in the exemplary apparatus 300 illustrated in FIGS. 11-14, measuring chamber 318 is incorporated within the housing 344 of a water release valve 330. Release valve 330 includes valve member 334 for containing and then releasing a measured pulse of liquid water to flash steamer 30. Release valve housing 344 has an interior cavity 332 that is divided by valve member 334 that acts an outlet valve for measuring chamber 318. When valve member 334 is in the closed position, the upper portion 338 of cavity 332 forms measuring chamber 318 which is primarily defined by the surfaces of sidewalls 340 and top wall 342 of upper portion 338. When closed, valve member 334 forms the bottom of measuring chamber 318.

Housing 344 of release valve 330 has an inlet opening 348 for receiving water from water tank 302, and an outlet opening 350 leading to inlet 352 of the heated cavity 32 of flash steamer 30. Housing 344 may be mounted directly to the flash steamer inlet 352, or optionally is connected to flash steamer 30 by a suitable conduit (not shown). Release valve 330 includes a solenoid 354 for opening and closing release valve member 334.

The flow of water into and out of measuring chamber 318 is controlled by control 116. Control 116 communicates with valves 322 and 330 through cables 358 and 360, respectively. Control 116 is programmed to automatically cause the opening and closing of valves 322 and 330 to accomplish the filling of measuring chamber 318 with a measured amount of water, and thereafter the releasing of the measured amount of water as an injected pulse of water into flash steamer 30. In the embodiment of the present invention shown in FIGS. 11-14, flash steamer 30 operates in the same manner as previously described for apparatus 10. Briefly, flash steamer 30 receives an injected pulse of water, converts the injected water into steam which is injected through wand 28 into an edible material 12 contained in container 24.

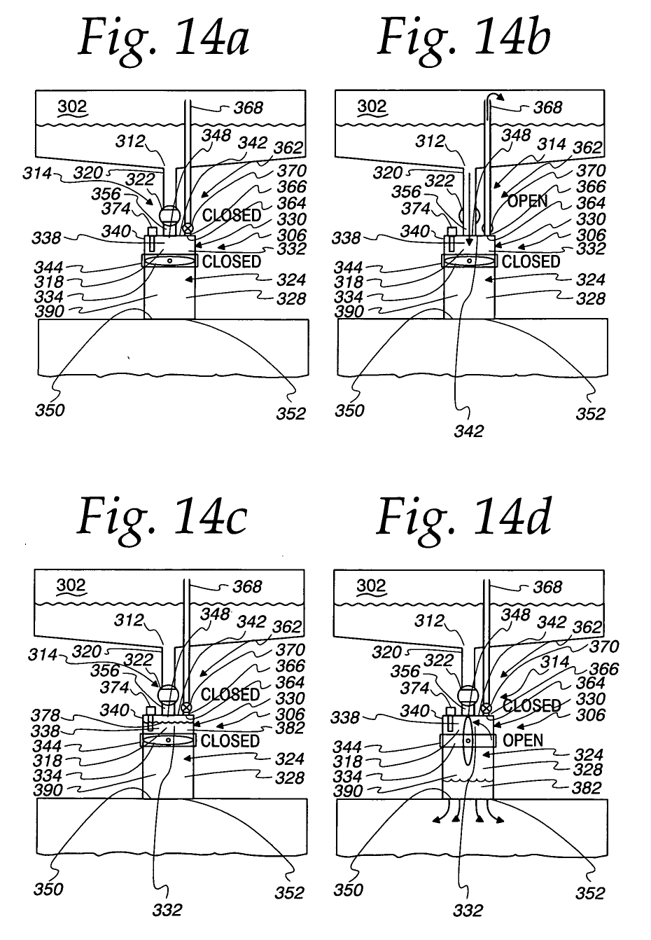

FIGS. 14a-d illustrate the stages of water flow into and out of measuring chamber 318 during a typical cycle for providing an injected pulse of water to flash steamer 30. FIG. 14a shows apparatus 300 in a resting or inactive position prior to the beginning of a water pulse providing cycle for cooking or heating. As shown in FIG. 14a, both inlet valve 322 and release valve 330 are in the closed position. At this point in time, measuring chamber 318 is empty, or at least substantially empty. It is being understood however, that there may be some small amount of residual water remaining in measuring chamber 318 from a prior cycle of releasing a pulse of water.

FIG. 14b illustrates the measuring chamber filling stage of the pulse providing cycle. To initiate filling, control 116 opens inlet valve 322 to allow water to flow by the force of gravity into water measuring chamber 318. In order to vent any air trapped in measuring chamber 318 during filling, an air venting means 362 may be provided. Exemplary air venting means 362 includes a measuring chamber venting outlet 364, a conduit 368 attached to venting outlet 364, and a solenoid venting valve 370. During water filling, venting valve 370 is opened by control 116 which communicates with venting valve 370 by cable 372. Any trapped air is allowed to vent through conduit 368 to a suitable location, such as water tank 302. After a period of time to allow for the suitable venting of trapped air, control 116 closes venting valve 370 before releasing the pulse of water from measuring chamber 318. Any other suitable alternative means for venting trapped air may be provided as is known to those skilled in the art. For example, venting valve 370 may be replaced with a valve that allows the venting of air, but not water, to the atmosphere. The venting of trapped air may also be accomplished, for example, by attaching a closed end conduit (not shown) to venting outlet 364 to provide a volume into which rising trapped air can accumulate.

A sensor for sensing the amount of water contained in measuring chamber 318, such as a water level sensor 374 is provided. Water level sensor 374 senses the water level 378 of the water contained in water measuring chamber 318, and sends the sensed information to control 116 by cable 376. During the water filling stage illustrated in FIG. 14b, inlet valve 322 remains in the open position to allow water to flow into measuring chamber 318. Control 116 receives the sensed water level information from water sensor 374, and is programmed to automatically close inlet valve 322 to provide a filled measuring chamber 318. A filled or full measuring chamber 318 means that water chamber 318 has been filled with a desired pre-selected and predetermined volume or amount of water. This predetermined amount of water is the amount of water that is later released as a measured pulse to flash steamer 30. It is noted that depending upon the predetermined amount of water that is desired and the volume of measuring chamber 318, a filled or full water chamber does not necessarily mean that measuring chamber 318 is filled to capacity. As explained below in greater detail, the level of water for a filled measuring chamber 318, could be at an intermediate height along the height of sidewall 340, or in some instances at a height above top wall 342. It is noted that the desired predetermined amount of water in a filled measuring chamber 318 may be different depending on the type of heating and amount of and type of edible material to be heated.

In one preferred embodiment of apparatus 300, water level sensor 374 continually senses the water level as it rises within measuring chamber 318. Alternatively, level sensor 374 may be limited to only providing a sensed indication as to whether or not the water level 378 has reached a predetermined height. In either case, once the water level reaches a certain predetermined level, a “water level attained signal” is sent from level sensor 374 to control 116, and control 116 thereafter automatically causes the closing of inlet valve 322.

Depending on the accuracy desired for the predetermined amount of the pulse, it may be necessary to consider the amount of post water flow through inlet valve 322. By post water flow it is meant the amount of water flow that occurs in the period of time after the water level attained signal is sent to control 116 and the point in time when inlet valve 322 is fully closed. The typical amount of post water flow can be determined and thereafter used for calibrating the operation of the system during the filling stage of the pulse providing cycle. Once the typical post flow amount is determined, the water level at which the water level attained signal is sent can be set taking into account the post flow amount. Stated another way, the water level at which the water level attained signal is sent is the water level at which chamber 318 contains an amount of water which is equal to desired predetermined amount of the water for the pulse minus the typical post water flow expected before inlet valve 322 is fully closed.

Another manner of achieving a desired predetermined amount of water for a pulse can be accomplished by filling measuring chamber 318 to full capacity. Used in this sense, a full capacity filling occurs when measuring chamber 318 can no longer accept additional water, i.e., even when inlet valve 322 is in the open position, no further flow through inlet valve 322 occurs. A filling to full capacity can be accomplished by opening inlet valve 322 for at least the typical period of time necessary for filling to a full capacity before automatically closing inlet valve 322. Another manner of filling to full capacity is to provide additional sensing means (not shown) that indicates when water flow through on opened inlet valve 322 has terminated, and then closing inlet valve 322.

One way to determine the typical amount of water contained in measuring chamber 318 when filled to capacity is by taking actual measurements of the amount of water released as a pulse after a filling to full capacity. Taking an actual measurement of the amount of water in a released pulse of capacity filling would take into consideration the volume of any trapped air and/or any water in measuring chamber 318 which extends to a position above top wall 342. For example, when filled to capacity, water may be contained in the lower portion 356 of upper conduit 320. Water may also be contained in conduit lower portion 366 of conduit 368 beneath venting valve 370 or other alternative venting means used. Alternatively, trapped air may cause the water level in measuring chamber 318 to be at a position lower than top wall 342. In any event, the typical amount of water contained in measuring chamber 318 that is filled to capacity will be relatively consistent and provide substantially equivalent pulse amounts of the desired predetermined amount. Thus, if the filling to capacity method of filling measuring chamber 318 is to be used, apparatus 300 and measuring chamber 318 are sized and configured so that when filled to capacity, measuring chamber 318 contains an amount of water that is the desired predetermined amount of water for the water pulse to be injected into flash steamer 30 for heating.

It can also be appreciated that if filling to capacity is the mode of operation for providing the measured pulse of water, it would be possible to operate apparatus 300 without using a water lever sensor 374. This is because during the filling of measuring chamber 318, inlet valve 322 is opened for a predetermined period of time, or alternatively until flow through the opened inlet valve 322 has been sensed to have ceased. In other words, to fill measuring chamber 318 to capacity, the closing of inlet valve 322 is not based on sensed water levels. Thus, providing a water level sensor 374 would not be required.

FIG. 14c illustrates one example of a water chamber 318 that has been filled. Control 116 has closed inlet valve 322 and measuring chamber 318 is now full, i.e., contains the desired predetermined amount of water for a pulse. During filling and prior to pulse release, control 116 maintains release valve 330 in the closed position. FIG. 14d illustrates the pulse releasing or pulse injecting stage of the water pulse providing cycle that follows the water filling stage shown in FIG. 14c, typically a short time thereafter. During the pulse injection stage, inlet valve 322 is in the closed position and control 116 has automatically opened release valve 330. Upon the opening of release valve 330, all of the water 382 contained in measuring chamber 318 is released to flow by the force of gravity and injected into flash steamer 30. Preferably, the releasing of water 382 is accomplished relatively rapidly, such as by quickly dumping the pulse. If the pulse of water is released too slowly, for example as trickle of water, the flow of water 382 out of measuring chamber 318 could be hindered. This is because pressure, resulting from steam formation as the released water contacts heated surface 128 of flash steamer 30, could hinder the continued flow of the water being released from measuring chamber 318.

Thus, apparatus 300 is preferably configured in order to accomplish a relatively rapid release or discharge of the water contained in measuring chamber 318 upon the opening of release valve 330. Preferably, measuring chamber 318 has a height and diameter that provides a relatively high diameter to height ratio, preferably a height to diameter ratio of about 2:1 or higher. Stated another way, which is especially applicable for a non-cylindrically shaped measuring chamber 318, the portion of the interior volume of measuring chamber 318 that is taken up by the water when measuring chamber 318 is filled, has a cross-sectional area at the outlet opening to volume ratio that is relatively high; preferably about 1:1 or higher.

Additionally, apparatus 300 is preferably configured and sized so that its downstream flowpath 328 is not overly restrictive for a pulse traveling therethrough. This may be accomplished by making the diameter of butterfly type valve member 334, the diameter for the lower portion 390 of cavity 332, the diameter for release valve outlet opening 350, and the diameter of flash steamer inlet 352 all approximately the same or greater than the diameter of water chamber 318.

Thus, in the preferred configuration of apparatus 300, a released pulse of water will preferably be of a shape which in general has a relatively high height to diameter ration, preferably about 2:1 or higher. Stated another way, the injected pulse traveling towards heated surface 128 of flash steamer 30 has a relatively high cross-sectional area to volume ratio, preferably about 1:1 or higher. Doing so lowers the duration of time from the initial contact of the water pulse with flash steamer heated surface 128, until the point in time that substantially all the released water pulse has reached heated surface 128. This, in turn, reduces the chance that the pressure created by the generated steam will substantially restrict the flow of the pulse of water as it is being released from water measuring chamber 318.

It is noted that during the pulse releasing stage of the pulse providing cycle shown in FIG. 14d, inlet valve 322 is closed. This prevents pressurized steam, water or air from flowing upward into water tank 302. Additionally, control 116 may be programmed to close release valve member 334 shortly after all of the pulse of water from measuring chamber 318 has flowed downstream of release valve member 334. This, for example, could be accomplished by determining the amount of time it typically takes for a released pulse to pass through an opened release valve member 334, and timing the opening and closing of release valve 334 accordingly. Quickly closing valve member 334 after the pulse has passed therethrough would act to protect water level sensor 374 from the possibly deleterious effects of pressurized steam flowing up from flash steamer 30 and into measuring chamber 318.

Having described the operational cycle for providing a measured water pulse, a more specific description of the operation of apparatus 300 during the heating of a liquid or solid edible material will now be provided. The operation of apparatus 300 during cooking will be described first, and then the operation for thermalizing. The operation of apparatus 300 for cooking a single-sized serving of edible material with a single pulse of injected water is similar to that previously described for the operation of apparatus 10 for cooking a single-sized serving with a single pulse of injected water. To cook a predetermined single-sized serving of scrambled eggs, for example, a single-sized serving amount of liquid egg 12 is placed in container 24. Container 24 is then mounted to apparatus 300. A safety interlock sensor (not shown) will indicate to control 116 if container 24 is properly secured. If not, operation will not begin until container 24 is mounted properly. The user then enters the appropriate selections for cooking a single-sized serving of scrambled eggs. Scrambled eggs is selected as the edible material type by using food type selector input 398. A single-size serving is entered by depressing button 138 marked “1”. Optionally, the selections that have been made by the user may be displayed for viewing at a display 392. Before injecting a pulse of water, control 116 determines whether the flash steamer 30 has been heated to a sufficient temperature for operation. If not, control 116 delays starting the cooking until flash steamer 30 is sufficiently preheated. If the temperature of flash steamer 30 is sufficient, control 116 thereafter automatically begins the pulse providing cycle as illustrated in FIGS. 14a-d, and as previously described in detail. This includes the filling of measuring chamber 318 with a predetermined amount or volume of water and thereafter releasing a measured pulse of water. In the present example, the predetermined amount of the measured pulse of water would be that for cooking a single-sized serving of scrambled eggs. More specifically, the predetermined pulse size is the predetermined amount of injected water that, when injected into heated cavity 32 and substantially all of the injected water is converted into steam, is sufficient to produce a quantity of steam that is sufficient for cooking a single-sized serving of scrambled eggs.

In a similar manner, apparatus 300 may also be used to cook single-sized portions of another type of liquid or solid edible material, for example, 6 ounces of noodles, or 6 ounces of soup. To do so, the appropriate food type is inputted at food type selector 398 and single-sized serving is selected by depressing button 138 marked “1”. Control 116 will then automatically fill measuring chamber 318 during the filling stage of the pulse providing cycle as discussed in detail previously in connection with FIGS. 14a-d. Specifically, control 116 causes measuring chamber 318 to be filled with the specific predetermined amount of water for cooking a single-sized serving of the selected type of liquid or edible material. Thus, for cooking a single-sized serving of noodles for example, a filled measuring chamber 318 contains the predetermined amount of water that, when injected into heated cavity 32 and substantially all of the injected water is converted into steam, is sufficient to produce a quantity of steam that is sufficient for cooking a single-size serving of noodles.

In can be appreciated that cooking different types of single-sized servings may require different pulse sizes, including possibly relatively substantially different amounts. In order to provide flexibility in pulse sizes, apparatus 300 can be calibrated and programmed to allow filling measuring chamber 318 to different predetermined water levels before closing inlet valve 322. For illustrative purposes only, measuring chamber 318 could be filled to 25%, 50% or 75% of capacity for respectively cooking a single-sized serving of soup, noodles or scrambled eggs. Thus, apparatus 300 is programmed to automatically fill measuring chamber 318 to the water level that provides the desired predetermined amount of water for an injected pulse for the specific type of edible material selected at food type selector input 398.

It would also be possible to provide different sized water pulse amounts by modifying apparatus 300 to include more than one water measuring chamber 318. In such a modified apparatus 300, each measuring chamber 318 is provided with a different volume that is specially sized for cooking a single-sized serving of a type of liquid or solid edible food. For example, one measuring chamber 318 when filled to capacity would contain the amount of water that is sufficient for cooking a single-sized serving of scrambled eggs, while another measuring chamber 318 is sized so that when filled to capacity it contains an amount of water for cooking a single-sized serving of noodles. The multiple measuring chambers (not shown) may be stacked vertically in series along flowpath 314 between water tank 302 and flash steamer 30. Alternatively, the multiple measuring chambers 318 could be positioned in a parallel arrangement and could also have separate inlets into flash steamer 30. With a modified apparatus 300 with multiple measuring chambers 318, control 116 is programmed to automatically select usage and filling of the appropriate water measuring chamber 318 depending upon the type of edible material selected by a user at food type input 398.

Like apparatus 10, apparatus 300 is also readily adaptable for cooking integer multiples of single-sized servings of edible material placed in container 24. This is accomplished by apparatus 300 sequentially repeating the cycle of filling and releasing water from measuring chamber 318. For example, if the amount of liquid eggs placed in container 24 is for three times the amount of a single-serving, the user would input scrambled eggs at food type selector input 398 and then press the “3” serving button 160. Control 116 would then automatically sequentially provide three pulse providing cycles. Each injected pulse would be provided by a separate pulse providing cycle as illustrated in FIGS. 14a-d. The amount of water in each pulse would be the predetermined amount of water for cooking a single-sized serving of scrambled eggs. Each injected pulse of water injected into flash steamer 30 produces steam that is injected through wand 28 into the edible material 12 held in container 24.

Stated in greater detail, to cook an amount of edible material that is three times a single-sized serving, control 116 first causes valve 330 to close, if not already closed, and then opens valve 322 to fill measuring chamber 318 with the predetermined amount of water for an injected pulse for the specific type of edible material selected at food type input 398. Thereafter, control 116 closes inlet valve 322 and opens release valve 330 to inject the first pulse of water from measuring chamber 318 into the flash steamer 30. Once valve 330 has been left open a predetermined period of time which is known to be long enough to release all of the water in measuring chamber 318, control 116 automatically provides a second filling of water to measuring chamber 318. Once again, this is accomplished by closing release valve 330 and opening inlet valve 322 to allow water to flow into measuring chamber 318 until filled. Thereafter, control 116 releases a second pulse to flash steamer 30 by once again closing inlet valve 322 and opening valve 330. Control 116 then automatically provides a third and final pulse of water by closing valve 330 and opening valve 322 to allow filling of water measuring chamber 318, and thereafter closing inlet valve 322 and opening valve 330 to release the water contained in water measuring chamber 318 as the third injected pulse.

Apparatus 300 may also be used for thermalizing liquid or solid edible materials. The thermalizing of the edible material may be thermalizing to a pre-selected elevated temperature, or alternatively may be thermalizing for a selected period of time. The amount of edible material that can be thermalized is not limited to that of a single-sized serving, or integer multiples thereof, but may be any amount that is within the capacity limitations of container 24. Thermalizing with apparatus 300 is similar to that described in connection with apparatus 10, but rather than the liquid water injector being solenoid 120, the liquid water injector means of apparatus 300 comprises the operation of measuring water 318 through the pulse providing cycles as shown in FIGS. 14a-d.

Thus, to thermalize a quantity of liquid or solid edible material, the edible material is placed in container 24 and mounted to apparatus 300. To thermalize to a desired temperature, the temperature is inputted by the user at temperature selection buttons 170 and 172. The inputted temperature is displayed on display 174. Start steaming button 176 is pushed to begin thermalization. Thereafter, control 116 sequentially causes individual pulses of water to be injected into flash steamer 30. Each pulse is provided by a pulse providing cycle as described previously and shown in FIGS. 14a-d. The size of each pulse provided for thermalization, typically will be less than a pulse size provided for cooking a single-sized serving with a single injected pulse of water. The smaller sized pulse amounts can be accomplished, for example, by filling measuring chamber 318 to a relatively lower water level during the filling stage illustrated in FIG. 14b, and then automatically closing inlet valve 322. Alternatively, smaller volume pulses could be accomplished by providing apparatus 300 with a second smaller volume measuring chamber 318 (not shown). Such second smaller measuring chamber 318 has a volume that when filled to full capacity provides the smaller desired predetermined amount of water for an injected pulse for thermalizing. The second smaller measuring chamber 318 could be arranged in series with the other measuring chamber 318 used for cooking, or could be arranged in parallel with each measuring chamber 318 having its own inlet into flash steamer 30.

The smaller volume of sequentially injected water pulses allows the temperature of the edible material being thermalized to be incrementally elevated in temperature as apparatus 300 provides sequential cycles of steam injection heating through wand 28. Each injected pulse of water is formed and injected in the manner previously described and illustrated in FIGS. 14a-d. Each injected pulse of water in turn is used to form an amount of steam at flash steamer 30 that is then injected into the edible material being heated in container 24 through wand 28. It is noted that for thermalizing, the consistency of the amount of each water pulse released from measuring chamber 318 is not as critical as cooking specific single-sized servings with a single pulse. As described previously regarding thermalizing to a pre-selected temperature using apparatus 10, the temperature of the edible material in container 24 is sensed at temperature probe 164 and communicated to control 116 through cable 186. After the sensed temperature of the material is elevated to the pre-selected inputted temperature, control 116 ceases providing injected water pulses to flash, steamer 30, and ceases the injection of steam into container 24.

Apparatus 300 may also be used to thermalize a liquid or solid edible material for a selected period of time, rather than thermalizing to a pre-selected elevated temperature. To do so, a user inputs the desired heating time mode button 175. Apparatus 300 then thermalizes the edible material placed in container 24 for the selected period of time by sequentially providing pulses of injected water from measuring chamber 318. After the selected period of time has elapsed, control 116 ceases operation of apparatus 300.

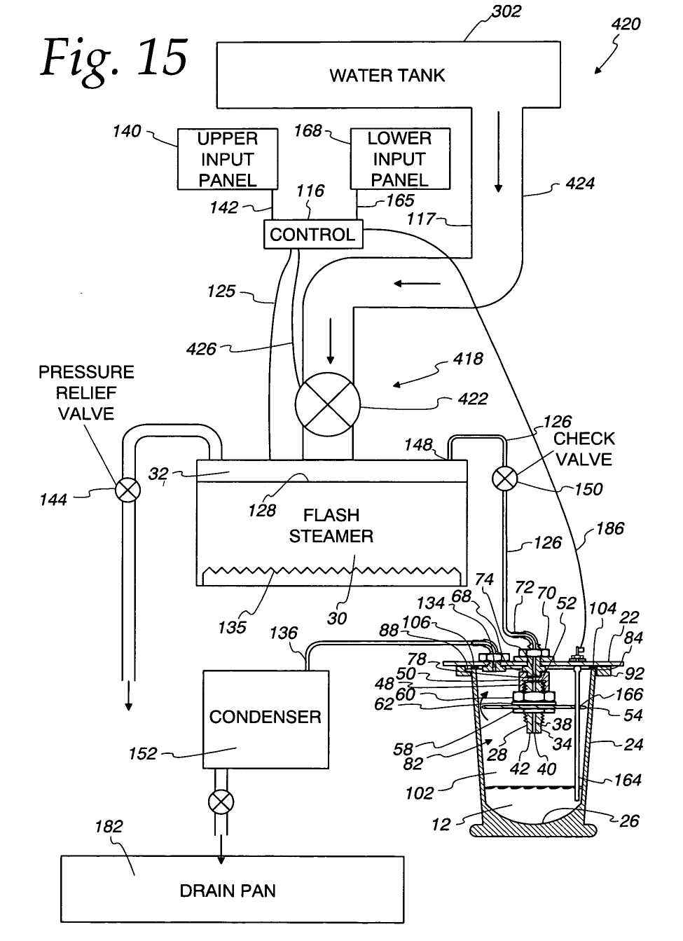

Another embodiment of the present invention is the apparatus 420 illustrated in FIG. 15. Apparatus 420, like apparatus 300 is used for heating liquid and solid edible materials. Apparatus 420 is similar to apparatus 300, except that apparatus 420 uses a different liquid water injector 418 to provide an injected water pulse of a predetermined amount. Rather than using liquid water injector 306 comprising measuring chamber 318 and valves 322, 330, liquid water injector 418 of apparatus 420 comprises a single valve 422. As described below in greater detail, to provide a water pulse of a predetermined amount, valve 422 is opened to initiate water flow and then automatically closed after a predetermined amount of water has flowed therethrough. This predetermined amount of water provides the pulse of water that is injected into flash steamer 30.

In the operation of apparatus 420, water from water tank 302 is fed by gravity through a conduit 424 that connects water tank 302 with valve 422. Conduit 424 is provided with a relatively large diameter that is capable of supplying water at a flow rate that is at least as great as the flow rate capable through an opened valve 422. Valve 422 communicates with control 116 through cable 426. Control 116 automatically opens valve 422 when a pulse is required for injection into flash steamer 30. In one preferred embodiment of apparatus 420, valve 422 is a flow control valve that regulates the flow rate through valve 422 so as to be at a substantially constant known flow rate. Achieving a desired predetermined amount of water for an injected pulse can be accomplished by control 116 automatically opening valve 422 to allow water to flow at the known flow rate of flow control valve 422 for a predetermined amount of time. The period of time that valve 422 is opened is the amount of time when multiplied by the flow rate of flow control valve 422 equals the desired predetermined amount of the water for the injected pulse. After that time period has elapsed, control 116 automatically closes valve 422.

In a manner similar to the previously described operation of apparatus 300, apparatus 420 may be utilized for cooking a single-sized serving of a liquid or edible material, integer multiples of single-sized servings, or for thermalizing various amounts of liquid or solid edible materials placed in container 24. For cooking a single-sized serving of an edible material, flow control type valve 422 is opened for the predetermined amount of time that provides a pulse of injected water of the desired predetermined amount to cook a single-sized serving of the type of edible material placed in container 24 and selected at food type input 398. For cooking a single-sized serving of noodles, for example, the predetermined amount of water is that amount, that when injected into heated cavity 32 and substantially all of the injected water is converted into steam, is sufficient to produce a quantity of steam that is sufficient for cooking a single-size serving of noodles. Control 116 is programmed with the appropriate time period needed to provide the predetermined amount of water for single-sized servings of each type of edible material selectable at food type selector input 398.

In a similar manner as described in connection with apparatus 300, apparatus 420 may be used to cook integer multiples of singled-sized servings of edible material. When an integer multiple of a single-sized serving is selected to be cooked, control 116 automatically sequentially opens flow control type valve 422 for the corresponding integer number of times to thereby sequentially inject the integer number of liquid pulses into flash steamer 30. Each injected liquid water pulse is converted into steam and injected into container 24.

Apparatus 420 may also be used for thermalizing a liquid or solid amount of edible material, either to a pre-selected temperature or for a pre-selected period of time. For thermalizing, valve 422 sequentially provides predetermined amounts of water for each injected pulse of liquid water that preferably are smaller than the amount of injected water pulse used to cook a single-size serving. The smaller pulse amounts allow an incremental increase in temperature of the material being thermalized. The smaller pulse amounts may be accomplished in the case of a flow control type valve 422, by opening valve 422 a shorter period of time than when cooking a single-size serving.

Thermalizing using apparatus 420 may be automatically conducted for a pre-selected period of time, or be automatically conducted until a pre-selected elevated temperature is achieved for the edible material placed in container 24. To thermalize for a selected period of time, the user depresses time mode button 175 and inputs the desired time period for thermalizing using up and down input buttons 170, 172, respectively, until the desired time appears at lower panel display 174. The user then depresses the start steaming button 176 to begin thermalization. During thermalization, control 116 automatically controls valve 422 to sequentially provide injected pulses of liquid water to flash steamer 30, and flash steamer 30 likewise sequentially injects steam through wand 28 into container 24 and edible material 12 contained therein. After the selected period of time ends, control 116 ceases providing injected pulses of liquid water from valve 422, and ceases causing steam to be injected from flash steamer 30 into container 24.

If a user desires to thermalize an edible material 12 to a pre-selected elevated temperature, the user presses a temperature mode button which can be a separate control button, or for example, button 175 programmed to set a temperature set point by pressing it twice and inputs the desired temperature using up and down inputs 170, 172, respectively, until the desired temperature appears in lower panel display 174. After the user pushes the start steaming button 176, control 116 causes valve 422 to automatically sequentially provide injected water pulses to flash steamer 30. Each injected pulse of water in turn causes steam to be formed by flash steamer 30 and to be sequentially injected into container 24. As discussed previously, temperature sensor 164 sends control 116 the sensed temperature of the edible material 12 being thermalized. Once the pre-selected elevated temperature has been achieved for the edible material 12 in container 24, control 116 automatically causes valve 422 to cease providing liquid water pulses to flash steamer 30, and in turn causes flash steamer 30 to cease injecting steam from flash steamer 30 into container 24.

Alternatively, the liquid water injector 418 of apparatus 420 could be a valve 422 that is a metering or dispensing type valve. When a metering type valve 422 is opened, valve 422 allows a preset and predetermined amount of water to flow through valve 422 before automatically closing. When used for cooking a single-sized serving, a metering valve 422 would be selected that dispenses a metered amount of water that is the predetermined amount of water that, when injected as a pulse of liquid water into flash steamer 30, and when converted to steam at flash steamer 30 is sufficient to cook the single-sized serving of the edible material selected. Apparatus 420 may be provided with multiple metering valves 422 (not shown) with each valve 422 set to dispense a metered predetermined amount of water sufficient for cooking a single-sized serving of a particular edible material. Apparatus 420 may also be provided with a separate metering type valve for providing a desired smaller sized injected pulse that is suitable for thermalizing for a period of time or to a pre-selected temperature. Alternatively, metering valve 422 may be automatically adjustable by control 116 to provide injected pulses of different predetermined amounts of water for cooking single-sized servings of different types of edible materials; and also be automatically adjustable to provide a desired smaller injected pulse amount for thermalizing edible materials. In such case, control 116 is programmed to automatically adjust metering valve 422 so as to provide the amount of liquid water to be dispensed as the injected pulse from metering valve 422. The amount of water injected by valve 422 would be based on the food type selected by the user at food type selector 398; or instead based on a user selection to thermalize an edible material, in which case a suitable relatively smaller injected pulse amount is provided by valve 422. When an integer multiple of a single-sized serving is selected to be cooked, control 116 automatically sequentially operates metering valve 422 to sequentially inject the corresponding integer number of injected water pulses from valve 422 to flash steamer 30.

While the invention has been described with respect to certain preferred embodiments, it is to be understood that the invention is capable of numerous changes, modifications and rearrangements without departing from the scope or spirit of the invention as defined in the claims.

What is claimed is:

1. An apparatus for cooking scrambled eggs comprising:
a water tank for containing a supply of liquid water,
a steam generator having a cavity for generating steam from liquid water injected into the steam generator;
a liquid water injector connectable to the supply of liquid water contained in the water tank for injecting a pulse of a predetermined amount of liquid water into the cavity of the steam generator, the predetermined amount of liquid water injected being sufficient so that when substantially all of the injected water is converted into steam the converted steam is sufficient to cook a predetermined quantity of liquid egg;
a container for containing at least a predetermined quantity of liquid egg;
a wand having a steam exit port positionable in the container with the steam exit port positioned proximate to the liquid egg, the wand being in fluid communication with the steam generator to receive steam generated therefrom for injecting into the liquid egg placed in the container to make scrambled eggs.

2. The apparatus for cooking scrambled eggs as claimed in claim 1 wherein the liquid water injector comprises a measuring chamber having an inlet and an outlet, the inlet connected to the water tank and the outlet connected to the steam generator, the measuring chamber for temporarily containing a predetermined amount of liquid water received from the water tank for subsequent injection.

3. The apparatus for cooking scrambled eggs as claimed in claim 1 wherein the liquid water injector comprises a flow control valve having an inlet in fluid communication with the water tank and an outlet in fluid communication with the steam generator, the flow control valve for providing the pulse of liquid water injected into the cavity of the steam generator by opening the flow control valve to initiate flow of the pulse and then closing the flow control valve after the predetermined amount of liquid water has flowed through the flow control valve.

4. The apparatus for cooking scrambled eggs as claimed in claim 1 further comprising a pump for pressurizing the supply of liquid water.

5. The apparatus for cooking scrambled eggs as claimed in claim 2 wherein the measuring chamber having a volume capacity for receiving liquid water, and the volume capacity of the measuring chamber is substantially the same as the volume of the predetermined amount of liquid water injected into the cavity as the pulse.

6. The apparatus for cooking scrambled eggs as claimed in 2 further comprising:
an inlet valve that is openable to allow water to flow from the water tank into the measuring chamber and is closable to prevent fluid flow between the measuring chamber and the water tank;
an outlet valve closable to contain water in the measuring chamber and openable to release the water contained in the measuring chamber for the pulse; and
a control for automatically opening the inlet valve and automatically closing the outlet valve to allow the predetermined amount of water to fill the measuring chamber, and thereafter opening the outlet valve to inject the predetermined amount of water as a pulse into the steam generator.

7. The apparatus for cooking scrambled eggs as claimed in claim 6 further comprising:
a sensor for sensing the amount of water contained in the measuring chamber and sending the sensed water amount information to the control to allow the control to automatically close the inlet valve to cause the amount of liquid water contained in the measuring chamber to be the predetermined amount of liquid water of the injected pulse.

8. An apparatus for cooking a food product comprising:
a steam generator having a cavity for generating steam from liquid water injected into the steam generator;
a liquid water injector connectable to a supply of liquid water, the liquid water injector including a measuring chamber for containing a predetermined amount of liquid water for injection of the contained predetermined amount of liquid water as a pulse of the predetermined amount of liquid water into the cavity of the steam generator, the predetermined amount of liquid water injected being sufficient so that when substantially all of the predetermined amount of water is converted into steam, the converted steam is sufficient to cook a predetermined quantity of the food product;
a container for containing at least a predetermined quantity of the food product placed therein;
a wand having a steam exit port positionable in the container with the steam exit port positioned proximate to the liquid or solid edible material, the wand being in fluid communication with the steam generator to receive steam generated therefrom for injection into the food product placed in the container to cook the predetermined quantity of the liquid or solid edible material.

9. The apparatus for cooking a food product as claimed in claim 8 wherein the amount of liquid water contained in the measuring chamber when filled to capacity is substantially the same as the predetermined amount of liquid water injected as a pulse into the cavity of the steam generator.

10. The apparatus for cooking a food product as claimed in claim 8 further comprising an inlet valve movable between an open and a closed position and an outlet valve moveable between an open and a closed position, a control for automatically controlling the opening and closing of the inlet valve and the outlet valve, the control automatically closing the outlet valve and opening the inlet valve to allow the filling and containing of water in the measuring chamber, and the control automatically closing the inlet valve and opening the outlet valve to inject the water contained in the measuring chamber as the injected pulse of water.

11. The apparatus for cooking a food product as claimed in claim 8 wherein the measuring chamber has a diameter to height ratio of about 2:1 or higher.

12. An apparatus for heating a food product comprising:
a steam generator for generating steam by injecting pulses of water onto a heated surface;
a wand in fluid communication with the steam generator, the wand having an exit port for injecting steam therethrough;
a container having an interior for a receiving a quantity of a food product to be heated;
a sealing surface for substantially sealing the container in a substantially closed environment;
mounting structure for removably mounting the container in a mounted position relative to the wand so that when the container is in the mounted position the exit port of the wand can be positioned proximate to the liquid or solid edible material and the interior of the container is substantially sealed by the sealing surface; and
a control for causing the injection of the pulses of water onto the heated surface of the steam generator.

13. The apparatus for heating a food product as claimed in claim 12 further comprising a measuring chamber for receiving and temporarily containing a predetermined amount of liquid water in the measuring chamber for injection as the injected pulse to the steam generator.

14. The apparatus for heating a food product as claimed in claim 12 further comprising:
a supply of liquid water;
a flow control valve having an inlet in fluid communication with the supply of liquid water and an outlet in fluid communication with the steam generator, the flow control valve opening to initiate the flow of liquid water to the steam generator and then closing after a predetermined amount of liquid water has flowed through the flow control valve to thereby provide the injected pulse of water.

15. The apparatus for heating a food product as claimed in claim 12 further comprising a tank for containing a supply of liquid water for providing the water for the pulses of water, and a pump for pressurizing the supply of liquid water.

16. The apparatus for heating a food product as claimed in claim 12 further comprising:
a temperature sensing device for sensing the temperature of the quantity of edible material in the container and sending the sensed temperature to the control,
the control sequentially injecting pulses of water onto the heated surface of the steam generator until the sensed temperature of the quantity of edible material is elevated to a predetermined temperature and thereafter ceasing the injection of pulses onto the heated surface.

17. The apparatus for heating a food product as claimed in claim 14 further comprising:
a timer device for selecting a period of time for the heating of the edible material by the apparatus;
the control automatically causing the apparatus to sequentially inject pulses of water onto the heated surface of the steam generator during the selected period of time and at the expiration of the period of time automatically ceasing the injection of pulses onto the heated surface.

18. A method of heating food comprising:
providing a water tank for containing a supply of water;
providing a container for holding food;
placing a quantity of food in the container;
positioning a wand having a terminal end proximate to but not in contact with the food;
sequentially injecting pulses of water from the water tank into a heated cavity of a steam generator to generate steam in the heated cavity from the pulses of the water injected into the heated cavity;
injecting the steam from the steam generator through the wand and into the food in the container;
automatically sensing the temperature of the food;
automatically terminating the steam injection into the food when a predetermined temperature of the food is reached.

19. The method of heating food as claimed in claim 18 further comprising:
providing a measuring chamber having a volume for receiving and containing water from the water tank;
filling the measuring chamber with water received from the water tank;
containing the water in the filled measuring chamber; and
injecting substantially all the water contained in the filled measuring chamber to provide one of the pulses of water injected into the heated cavity of the steam generator.

20. The method of heating food as claimed in claim 19 further comprising:
providing a water amount sensor for sensing the amount of water contained in the measuring chamber during filling;
sensing the amount of water contained in the measuring chamber during the filling of the measuring chamber; and
using the sensed information to automatically stop the filling of the measuring chamber during filling to provide a predetermined amount of water contained in a filled measuring chamber for injection as one of the pulses of water injected into the steam generator.

21. The method of heating food as claimed in claim 19 further characterized by filling the measuring chamber to full capacity and releasing the water in a filled measuring chamber to provide each pulse of water.

22. The method of heating food as claimed in claim 18 further comprising:
providing a valve positioned between the water tank and the heated cavity;
automatically opening the valve to allow a predetermined amount of water to flow through the valve to provide each of the pulses of injected water before automatically closing the valve.

23. The method of heating food as claimed in claim 22 wherein the valve is a metering valve.

24. The method of heating food as claimed in claim 22 wherein the valve is a flow control valve.

25. The method of heating food as claimed in claim 22 wherein the valve is a solenoid valve and the supply of water to the valve is pressurized.

26. The method of heating food as claimed in claim 19 wherein the pulse of injected water is formed in a shape that has cross-sectional area to volume ratio of about 1:1 or higher.

27. A method of cooking a food product comprising:
providing a predetermined amount of the food product in a container;
injecting a predetermined amount of water into a heated cavity;
forming a quantity of steam in the cavity from substantially all of the water injected into the cavity; and
injecting the steam into the container having the food contained therein wherein the quantity of steam is sufficient to cook the predetermined quantity of the liquid or solid edible material.

28. The method of cooking a liquid or solid edible material as claimed in claim 27 further comprising:
providing a measuring chamber for containing water;
supplying water to the measuring chamber so that the measuring chamber contains the predetermined amount of water; and
injecting the predetermined amount of water from the water contained in the filled measuring chamber into the heated cavity.

29. The method of cooking a liquid or solid edible material as claimed in claim 28 further characterized by the measuring chamber is filled to capacity during the supplying of water to the measuring chamber and the amount of water in the measuring chamber when filled to capacity is substantially the same as the predetermined amount of water injected into the cavity.

30. The method of cooking a liquid or solid edible material as claimed in claim 27 further comprising:
providing a flow control valve that is openable to allow water to flow therethrough and then automatically is closed after said predetermined amount of water has flowed through the flow control valve; and
supplying water to the flow control valve to inject said predetermined amount of water into the heated cavity.
